(19) United States
(12) Patent Application Publication
(10) Pub. No.: US 2026/0272822 A1
(43) Pub. Date: Sep. 17, 2026

(54) TOPICAL COMPOSITIONS CONTAINING TOFACITINIB AND IONIC LIQUID

(71) Applicant: CAGE BIO INC., San Carlos, CA (US)

(72) Inventor: Nitin JOSHI, San Carlos, CA (US)

(21) Appl. No.: 19/671,185

(22) Filed: May 7, 2026

Related U.S. Application Data
(63) Continuation of application No. PCT/US25/10627, filed on Jan. 7, 2025.
(60) Provisional application No. 63/618,374, filed on Jan. 7, 2024.

(51) Int. Cl. A61K 9/00; A61K 9/06; A61K 31/519; A61K 47/02; A61K 47/10; A61K 47/12; A61K 47/18; A61K 47/34

(52) U.S. Cl. CPC A61K 9/0014; A61K 9/06; A61K 31/519; A61K 47/02; A61K 47/10; A61K 47/12; A61K 47/186; A61K 47/34

(57) ABSTRACT

One aspect of the present disclosure is directed to a method of delivering tofacitinib to or through a skin. Another aspect of the present disclosure is directed to a method of locally inhibiting activity of at least one of JAK-1, JAK-2, TYK-2, and JAK-3 within an epidermis layer, a dermis layer, a subcutaneous tissue layer, or a muscle tissue. Another aspect of the present disclosure is directed to a topical composition, comprising tofacitinib or a pharmaceutically acceptable salt thereof, and an ionic liquid in an amount sufficient to allow a therapeutically effective amount of tofacitinib to reach a target depth within or beyond the skin.

### CROSS-REFERENCE TO RELATED APPLICATIONS

The present application is a continuation under 35 USC § 1.111(a) of International Patent Application PCT/US2025/010627, filed on Jan. 7, 2025, which claims priority to U.S. Provisional Patent Application 63/618,374, filed on Jan. 7, 2024. The prior applications are considered part of the disclosure of the present application and are incorporated herein by reference in their entirety.

### BACKGROUND OF THE DISCLOSURE

Tofacitinib, a Janus kinase (JAK) inhibitor, is sometime orally administered to treat rheumatoid arthritis, psoriatic arthritis and ulcerative colitis.

### SUMMARY OF THE DISCLOSURE

One aspect of the present disclosure is directed to a method of delivering tofacitinib to or through a skin. Another aspect of the present disclosure is directed to a method of locally inhibiting activity of at least one of JAK-1, JAK-2, TYK-2, and JAK-3 within an epidermis layer, a dermis layer, a subcutaneous tissue layer, or a muscle tissue. Another aspect of the present disclosure is directed to a topical composition, comprising tofacitinib or a pharmaceutically acceptable salt thereof, and an ionic liquid in an amount sufficient to allow a therapeutically effective amount of tofacitinib to reach a target depth within or beyond the skin.

Disclosed herein, in certain embodiments, is a method of delivering tofacitinib to or through a skin, the method comprising administering to the skin a pharmaceutical composition comprising tofacitinib or a pharmaceutically acceptable salt thereof, and an ionic liquid in an amount sufficient to allow a therapeutically effective amount of tofacitinib to reach a target depth within or beyond the skin, wherein the ionic liquid comprises a choline cation and a fatty acid anion.

Disclosed herein, in certain embodiments, is a method of locally inhibiting activity of at least one of JAK-1, JAK-2, TYK-2, and JAK-3 within an epidermis layer, a dermis layer, a subcutaneous tissue layer, or a muscle tissue, the method comprising administering to the skin a pharmaceutical composition comprising tofacitinib or a pharmaceutically acceptable salt thereof, and an ionic liquid in an amount sufficient to allow a therapeutically effective amount of tofacitinib to reach a target depth within or beyond the skin, wherein the ionic liquid comprises a choline cation and a fatty acid anion.

Disclosed herein, in certain embodiments, is a topical composition, comprising tofacitinib or a pharmaceutically acceptable salt thereof, and an ionic liquid in an amount sufficient to allow a therapeutically effective amount of tofacitinib to reach a target depth within or beyond the skin, wherein the ionic liquid comprises a choline cation and a fatty acid anion. In some embodiments, the topical composition is an ointment. In some embodiments, the topical composition is a gel.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates minipig PK study comprising skin and systemic exposure of tofacitinib after topical or oral administration;

FIG. 2 illustrates minipig PK study comprising skin and systemic exposure of tofacitinib after topical or oral administration;

FIG. 3 illustrates minipig PK study comprising skin and systemic exposure of tofacitinib after topical or oral administration;

FIG. 4 illustrates skin flux assay evaluation of a topical tofacitinib composition disclosed herein;

FIG. 5 illustrate the result of a Phase IIa clinical study based on a topical tofacitinib composition disclosed herein; and

FIG. 6 illustrate the result of a Phase IIa clinical study based on a topical tofacitinib composition disclosed herein.

### DETAILED DESCRIPTION OF THE DISCLOSURE

Disclosed herein, in certain embodiments, is a topical composition, comprising tofacitinib or a pharmaceutically acceptable salt thereof, and an ionic liquid in an amount sufficient to allow a therapeutically effective amount of tofacitinib to reach a target depth within or beyond the skin, wherein the ionic liquid comprises a choline cation and a fatty acid anion.

### Certain Terminology

The terminology used herein is for the purpose of describing particular cases only and is not intended to be limiting. The below terms are discussed to illustrate meanings of the terms as used in this specification, in addition to the understanding of these terms by those of skill in the art. As used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions described herein are. The upper and lower limits of these smaller ranges may independently be included in the smaller ranges and are also encompassed within the methods and compositions described herein, subject to any specifically excluded limit in the stated range. Where the stated range includes one or both of the limits, ranges excluding either or both of those included limits are also included in the methods and compositions described herein.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker). Further, these terms refer to human or animal subjects.

“Treating” or “treatment” refers to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to be prevented. For example, a subject or mammal is successfully “treated” for rosacea, if, after receiving a therapeutic amount of a composition according to the methods of the present disclosure, the subject shows observable and/or measurable reduction in or absence of one or more of the following: reduction in the erythema; reduction in the appearance of red veins; papules, and pustules.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of the compound, age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

Unless defined otherwise, all technical and scientific terms used herein have the same meaning as commonly understood by one of ordinary skill in the art to which the methods and compositions described herein belong. Although any methods and materials similar or equivalent to those described herein can also be used in the practice or testing of the methods and compositions described herein, representative illustrative methods and materials are now described.

### Tofacitinib

3-((3R,4R)-4-Methyl-3-[methyl-(7H-pyrrolo[2,3-d]pyrimidin-4-yl)-amino]-piperidin-1-yl)-3-oxopropionitrile, i.e. tofacitinib, has the following structural formula

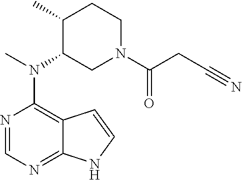 [SMILES: C[C@@H]1CCN(C(=O)CC#N)C[C@@H]1N(C)c1ncnc2[nH]ccc12]

The synthesis of 3-((3R,4R)-4-methyl-3-[methyl-(7H-pyrrolo[2,3-d]-pyrimidin-4-yl)-amino]-piperidin-1-yl)-3-oxopropionitrile is described in WO 2001/42246 and WO 2002/096909, commonly assigned to the assignee of the present invention and which are incorporated herein by reference in their entirety. The preparation of 3-((3R,4R)-4-methyl-3-[methyl-(7H-pyrrolo[2,3-d]-pyrimidin-4-yl)-amino]-piperidin-1-yl)-3-oxopropionitrile mono citrate salt is described in U.S. Pat. No. 6,965,027.

Tofacitinib and its pharmaceutically acceptable salt such as the mono citrate salt are also useful as inhibitors of protein kinases, such as the enzyme Janus Kinase (JAK). As such, tofacitinib and its pharmaceutically acceptable salt such as the mono citrate salt are sometimes orally administered therapy as immunosuppressive agents for organ transplants, xeno transplantation, lupus, multiple sclerosis, rheumatoid arthritis, psoriasis, Type I diabetes and complications from diabetes, cancer, asthma, atopic dermatitis, autoimmune thyroid disorders, ulcerative colitis, Crohn's disease, Alzheimer's disease, Leukemia and other indications where immunosuppression would be desirable. The present disclosure relates to administration of tofacitinib topically into or through the skin, which present unique challenges especially when the underlying diseases or conditions require delivery of therapeutically effective amount of tofacitinib at various target depths into or through the skin.

### Ionic Liquids

Described herein, in certain embodiments, are compositions comprising an ionic liquid comprising a choline cation and a fatty acid anion. In some embodiments, the composition further comprises a pharmaceutically acceptable solvent. In some embodiments, the fatty acid is myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid. In some embodiments, the fatty acid is geranic acid. In some embodiments, the fatty acid comprises 9 to 14 carbons. In some embodiments, the ionic liquid is liquid at room temperature. In some embodiments, the ionic liquid is liquid below 100° C.

In some embodiments, the ionic liquid is a deep eutectic solvent (DES). In some embodiments, a DES comprises excess carboxylate which precludes 1:1 ion pairing. In some embodiments, a DES further comprises a hydrogen-bond donor. In some embodiments, the hydrogen-bond donor is urea or citric acid. In some embodiments, the solvent properties of a DES are adjusted by changing the hydrogen-bond donor. In some embodiments, the ammonium salt of a DES interacts with a hydrogen-bond donor. In some embodiments, the DES has a melting point lower than either of the individual components (e.g. fatty acid and choline).

In some embodiments, the ionic liquid comprises a molar ratio of a choline cation to a fatty acid anion of 1:0.5 to 1:10. In some embodiments, the molar ratio of the choline cation to the fatty acid anion is about 1:0.5, 1:0.6, 1:0.7, 1:0.8, 1:0.9, 1:1.0; 1:1.1, 1:1.2, 1:1.3, 1:1.4, 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, 1:4.0, 1:4.1, 1:4.2, 1:4.3, 1:4.4, 1:4.5, 1:4.6, 1:4.7, 1:4.8, 1:4.9, 1:5.0, 1:5.1, 1:5.2, 1:5.3, 1:5.4, 1:5.5, 1:5.6, 1:5.7, 1:5.8, 1:5.9, 1:6.0, 1:6.1, 1:6.2, 1:6.3, 1:6.4, 1:6.5, 1:6.6, 1:6.7, 1:6.8, 1:6.9, 1:7.0, 1:7.1, 1:7.2, 1:7.3, 1:7.4, 1:7.5, 1:7.6, 1:7.7, 1:7.8, 1:7.9, 1:8.0, 1:8.1, 1:8.2, 1:8.3, 1:8.4, 1:8.5, 1:8.6, 1:8.7, 1:8.8, 1:8.9, 1:9.0, 1:9.1, 1:9.2, 1:9.3, 1:9.4, 1:9.5, 1:9.6, 1:9.7, 1:9.8, 1:9.9, or about 1:10. In some embodiments, the molar ratio of the choline cation to the fatty acid anion is about 1:1.1, 1:1.2, 1:1.3, 1:1.4, 1:1.5, 1:1.6, 1:1.7, 1:1.8, 1:1.9, or 1:2.0.

In some embodiments, the choline cation and fatty acid anion are in a molar ratio in the ionic liquid. In some embodiments, the choline cation and fatty acid anion are in a molar ratio of 1:1. In some embodiments, the term Composition B is used herein to refer to a composition or an ionic liquid comprising a 1:1 molar ratio of choline cation to geranic acid anion. In some embodiments, Composition B does not comprise water.

In other embodiments, the choline cation and fatty acid anion are in a molar ratio of 1:2. In some embodiments, the term Composition A is used herein to refer to a composition or an ionic liquid comprising a 1:2 molar ratio of choline cation to geranic acid anion. In some embodiments, Composition A does not comprise water.

In some embodiments, the chemical structure of choline is:

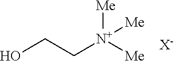

wherein X⁻ is a pharmaceutically acceptable anion.

In some embodiments, term choline refers to the class of quaternary ammonium salts containing the N,N,N-trimethylethanolammonium cation. In some embodiments, the X⁻ on the right of the structure of choline denotes a pharmaceutically acceptable anion. In some embodiments the X⁻ is bicarbonate, carbonate, acetate, citrate, tartarate, bitartarate, lactate, chloride, bromide, or iodide. In some embodiments, the X⁻ is bicarbonate. In some embodiments, the choline is an anti-inflammatory agent.

In some embodiments, choline is in the form of a pharmaceutically acceptable salt. The type of pharmaceutical acceptable salts, include, but are not limited to acid addition salts, formed by reacting the free base form of the compound with a pharmaceutically acceptable: inorganic acid such as hydrochloric acid, hydrobromic acid, sulfuric acid, nitric acid, phosphoric acid, metaphosphoric acid, and the like; or with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

In some embodiments, the chemical structure of geranic acid, or 3,7-dimethyl-2,6-octadienoic acid, is:

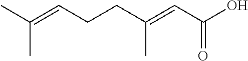

In some embodiments, geranic acid is in the form of a pharmaceutically acceptable salt. The type of pharmaceutical acceptable salts, include, but are not limited to salts formed when an acidic proton present in the parent compound either is replaced by a metal ion, e.g., an alkali metal ion (e.g. lithium, sodium, potassium), an alkaline earth ion (e.g. magnesium, or calcium), or an aluminum ion; or coordinates with an organic base. Examples of acceptable organic bases include, but are not limited to, ethanolamine, diethanolamine, triethanolamine, tromethamine, and N-methylglucamine. Examples of acceptable inorganic bases include, but are not limited to, aluminum hydroxide, calcium hydroxide, potassium hydroxide, sodium carbonate, and sodium hydroxide.

In some embodiments, the choline and the fatty acid are synthesized using any suitable standard synthetic reactions. In some embodiments, the reactions are employed in a linear sequence to provide the compounds or they may be used to synthesize fragments which are subsequently joined by any suitable method. In some embodiments, the starting material used for the synthesis of choline or fatty acid is synthesized or are obtained from commercial sources.

In some embodiments, geranic acid is purified from the commercially available technical grade (Sigma-Aldrich, St. Louis, Mo.) by repeated (5-7×) recrystallization from a solution of 70 wt % geranic acid/30 wt % acetone at −70° C. In some embodiments, purity of the geranic acid is assessed by 1H NMR spectroscopy and conductivity measurements. In some embodiments, the term geranic acid refers to a geranic acid or a salt thereof. In some embodiments, the geranic acid is an anti-microbial agent.

### Tofacitinib Topical Composition

In some embodiments, each component in a composition, such as the tofacitinib, ionic liquid, the pharmaceutically acceptable carrier, and optionally other components, is described a percent (%) of the composition. In some embodiments, the % of the composition is a percent concentration volume/volume (v/v) or a percent concentration weight/volume (w/v).

Disclosed herein, in certain embodiments, is a topical composition, comprising tofacitinib or a pharmaceutically acceptable salt thereof, and an ionic liquid in an amount sufficient to allow a therapeutically effective amount of tofacitinib to reach a target depth within or beyond the skin, wherein the ionic liquid comprises a choline cation and a fatty acid anion.

In some embodiments, the pharmaceutical composition comprises about 0.01% to about 10% of tofacitinib or a pharmaceutically acceptable salt thereof. In some embodiments, the pharmaceutical composition comprises about 0.1% to about 8% of tofacitinib or a pharmaceutically acceptable salt thereof. In some embodiments, the topical ointment comprises about 0.1% to about 5% of tofacitinib or a pharmaceutically acceptable salt thereof. In some embodiments, the target depth is from about 0.05 mm to about 20 mm. In some embodiments, the target depth is from about 0.1 mm to about 15 mm. In some embodiments, the target depth is from about 1 mm to about 10 mm

In some embodiments, the pharmaceutical composition allows therapeutically effective amount of tofacitinib to reach at least an epidermis layer. In some embodiments, the pharmaceutical composition allows therapeutically effective amount of tofacitinib to reach at least a dermis layer.

In some embodiments, the pharmaceutical composition allows therapeutically effective amount of tofacitinib to reach at least a subcutaneous tissue layer. In some embodiments, the pharmaceutical composition allows therapeutically effective amount of tofacitinib to reach at least a muscle tissue.

In some embodiments, the fatty acid is selected from the group consisting of myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, malonic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid. In some embodiments, the fatty acid is selected from the group consisting oleic acid, geranic acid, hexanoic acid, and malonic acid. In some embodiments, the fatty acid is geranic acid.

In some embodiments, the ionic liquid is a deep eutectic solvent (DES). In some embodiments, the ionic liquid comprises the choline cation and fatty acid anion in a molar ratio in a range of 1:1 to 1:4 of choline cation to fatty acid anion. In some embodiments, the ionic liquid comprises the choline cation and fatty acid anion in a molar ratio of 1:2 of choline cation to fatty acid anion.

In some embodiments, the pharmaceutical composition consists essentially of tofacitinib and the ionic liquid. In some embodiments, the pharmaceutical composition further comprises a pharmaceutically acceptable carrier. In some embodiments, the pharmaceutically acceptable carrier is an aqueous carrier. In some embodiments, the pharmaceutically acceptable carrier comprises an ointment base.

In some embodiments, the ionic liquid is present in the pharmaceutical composition at a concentration of from about 1% to about 99%. In some embodiments, the ionic liquid is present in the pharmaceutical composition at a concentration of from about 5% to about 90%. In some embodiments, the ionic liquid is present in the pharmaceutical composition at a concentration of from about 10% to about 80%. In some embodiments, the ionic liquid is present in the pharmaceutical composition at a concentration of from about 15% to about 70%. In some embodiments, the ionic liquid is present in the pharmaceutical composition at a concentration of from about 20% to about 60%. In some embodiments, the ionic liquid is present in the pharmaceutical composition at a concentration of from about 30% to about 50%. In some embodiments, the ionic liquid is present in the pharmaceutical composition at a concentration of about 35% to about 45%. In some embodiments, the ionic liquid is present in the pharmaceutical composition at a concentration of about 40%.

In some embodiments, the pharmaceutical composition further comprises a penetration enhancer. In some embodiments, the penetration enhancer is 2-(2-ethoxyethoxy) ethanol or oleyl alcohol. In some embodiments, the penetration enhancer is 2-(2-ethoxyethoxy) ethanol. In some embodiments, the pharmaceutical composition comprises from about 1% to about 20% of 2-(2-ethoxyethoxy) ethanol. In some embodiments, the pharmaceutical composition comprises from about 5% to about 15% 2-(2-ethoxyethoxy) ethanol. In some embodiments, the pharmaceutical composition comprises from about 10% 2-(2-ethoxyethoxy) ethanol.

In some embodiments, the pharmaceutical composition is essentially free of 2-(2-ethoxyethoxy) ethanol. In some embodiments, the pharmaceutical composition is essentially free of oleyl alcohol.

In some embodiments, the composition comprises the ionic liquid in a concentration of about 0.1% to 99%. In some embodiments, the composition comprises the ionic liquid in a concentration of about 1% to 40%. In some embodiments, the composition comprises the ionic liquid in a concentration of about 1% to 20%. In some embodiments, the composition comprises the ionic liquid in a concentration of about 5% to 20%. In some embodiments, the composition comprises the ionic liquid in a concentration of about 5% to 40%. In some embodiments, the composition comprises the ionic liquid in a concentration of about 20% to 40%. In some embodiments, the composition comprises the ionic liquid in a concentration of about 20% to 60%. In some embodiments, the composition comprises the ionic liquid in a concentration of about 20% to 80%.

In some embodiments, the composition comprises the ionic liquid in a concentration of about 0.1% to 99%, and the pharmaceutically acceptable solvent in a concentration of about 1% to about 99.9%. In some embodiments, the composition comprises the ionic liquid in a concentration of about 1% to 40%, and the pharmaceutically acceptable solvent in a concentration of about 60% to about 99%. In some embodiments, the composition comprises the ionic liquid in a concentration of about 20% to 40%, and the pharmaceutically acceptable solvent in a concentration of about 80% to about 99%. In some embodiments, the composition comprises the ionic liquid in a concentration of about 20% and the pharmaceutically acceptable solvent in a concentration of about 80%. In some embodiments, the composition comprises the ionic liquid in a concentration of about 40% and the pharmaceutically acceptable solvent in a concentration of about 60%.

In some embodiments, the composition comprises the ionic liquid in a concentration of about 1% to 50%, and the pharmaceutically acceptable solvent in a concentration of about 50% to 99%. In some embodiments, the composition comprises the ionic liquid in a concentration of about 1% to 50%, and water in a concentration of about 50% to 99%. In some embodiments, the water is deionized water or Milli-Q® water.

In some embodiments, the composition comprises the ionic liquid in a concentration of about 1% to 50%, a pharmaceutically acceptable solvent in a concentration of about 1% to 50%, and a gelling agent in a concentration of about 1 to 5%. In some embodiments, the composition comprises the ionic liquid in a concentration of about 1% to 50%, and water in a concentration of about 1% to 50%.

In some embodiments, the pharmaceutically acceptable solvent is diisopropyl adipate. In some embodiments, the composition comprises diisopropyl adipate in a concentration of about 20%. In some embodiments, the composition comprises the ionic liquid in a concentration of about 1% to 40%, and diisopropyl adipate in a concentration of about 60% to about 99%.

In some embodiments, the composition comprises a gel base in a concentration of about 50% to 90% of the composition. In some embodiments, the composition comprises a gel base in a concentration of about 50%, 60%, 70%, 80%, or 90% of the composition.

In some embodiments, preparing an ionic liquid comprising a choline cation and a fatty acid anion comprises: (a) mixing choline and a fatty acid in a solvent at room temperature in a predetermined ratio; and (b) removing the solvent in vacuo. In some embodiments, the fatty acid is geranic acid. In some embodiments, the solvent is water. In a particular embodiment, the water is deionized water. In some embodiments, removing the solvent comprises rotary evaporation. In some embodiments, removing the solvent comprises heating the ionic liquid, applying a vacuum to the ionic liquid, or a combination thereof. In some embodiments, preparing the ionic liquid further comprises drying the ionic liquid. In some embodiments, heating the ionic liquid comprises heating the ionic liquid to 60° C. In some embodiments, the heating is done for at least 10 minutes, 20 minutes, 30 minutes, 1 hour, 2 hours, 3 hours, 4 hours, 5 hours, 6 hours, 7 hours, 8 hours, 9 hours, 10 hours, 11 hours, 12 hours, 24 hours, 36 hours, 48 hours or 60 hours. In some embodiments, the vacuum is applied at −100 kPa. In some embodiments, the vacuum is applied for at least 10 minutes, 20 minutes, 30 minutes, 1 hours, 2 hours, 3 hours, 4 hours, 5 hours, 6 hours, 7 hours, 8 hours, 9 hours, 10 hours, 11 hours, 12 hours, 24 hours, 36 hours, 48 hours or 60 hours.

In some embodiments, the ionic liquid has had the solvent used in the ionic liquid preparation process removed. In some embodiments, the ionic liquid does not comprise water.

In some embodiments, choline is choline bicarbonate. In some embodiments, the choline is choline in an 80% wt solution of choline bicarbonate. In some embodiment, the predetermined ratio is a ratio of 1:1, 1:2, 1:3, or 1:4 of a choline cation: fatty acid anion. In one embodiment, the ratio is a molar ratio. In another embodiment, the ratio is ratio by weight.

In some embodiments, isolating the composition further comprises purifying the ionic liquid. In some embodiments, purifying the ionic liquid comprises using conventional techniques, including, but not limited to, filtration, distillation, crystallization, and chromatography. In some embodiments, preparing the ionic liquid further comprises isolating the purified ionic liquid

### Tofacitinib Topical Delivery

Disclosed herein, in certain embodiments, is a method of delivering tofacitinib to or through a skin, the method comprising administering to the skin a pharmaceutical composition comprising tofacitinib or a pharmaceutically acceptable salt thereof, and an ionic liquid in an amount sufficient to allow a therapeutically effective amount of tofacitinib to reach a target depth within or beyond the skin, wherein the ionic liquid comprises a choline cation and a fatty acid anion.

In some embodiments, the pharmaceutical composition comprises about 0.01% to about 10% of tofacitinib or a pharmaceutically acceptable salt thereof. In some embodiments, the pharmaceutical composition comprises about 0.1% to about 8% of tofacitinib or a pharmaceutically acceptable salt thereof. In some embodiments, the pharmaceutical composition comprises about 0.1% to about 5% of tofacitinib or a pharmaceutically acceptable salt thereof.

In some embodiments, the target depth is from about 0.05 mm to about 20 mm. In some embodiments, the target depth is from about 0.1 mm to about 15 mm. In some embodiments, the target depth is from about 1 mm to about 10 mm.

In some embodiments, the pharmaceutical composition allows therapeutically effective amount of tofacitinib to reach at least an epidermis layer. In some embodiments, the amount of tofacitinib at the epidermis layer is sufficient to inhibit activity of at least one of JAK-1, JAK-2, TYK-2, and JAK-3 within the epidermis layer.

In some embodiments, the pharmaceutical composition allows therapeutically effective amount of tofacitinib to reach at least a dermis layer. In some embodiments, the amount of tofacitinib at the dermis layer is sufficient to inhibit activity of at least one of JAK-1, JAK-2, TYK-2, and JAK-3 within the dermis layer.

In some embodiments, the pharmaceutical composition allows therapeutically effective amount of tofacitinib to reach at least a subcutaneous tissue layer. In some embodiments, the amount of tofacitinib at the subcutaneous tissue layer is sufficient to inhibit activity of at least one of JAK-1, JAK-2, TYK-2, and JAK-3 within the subcutaneous tissue layer.

In some embodiments, the pharmaceutical composition allows therapeutically effective amount of tofacitinib to reach at least a muscle tissue. In some embodiments, the amount of tofacitinib at the muscle tissue is sufficient to inhibit activity of at least one of JAK-1, JAK-2, TYK-2, and JAK-3 within the muscle tissue.

In some embodiments, the total amount of tofacitinib reaching at least the epidermis layer provided by the pharmaceutical composition is at least 1.5 times the total amount of tofacitinib reaching at least the epidermis layer provided by a control composition replacing the ionic liquid with same amount of 2-(2-ethoxyethoxy) ethanol. In some embodiments, the total amount of tofacitinib reaching at least the epidermis layer provided by the pharmaceutical composition is at least 2 times the total amount of tofacitinib reaching at least the epidermis layer provided by a control composition replacing the ionic liquid with same amount of 2-(2-ethoxyethoxy) ethanol. In some embodiments, the total amount of tofacitinib reaching at least the epidermis layer provided by the pharmaceutical composition is at least 2.5 times the total amount of tofacitinib reaching at least the epidermis layer provided by a control composition replacing the ionic liquid with same amount of 2-(2-ethoxyethoxy) ethanol. In some embodiments, the total amount of tofacitinib reaching at least the epidermis layer provided by the pharmaceutical composition is at least 3 times the total amount of tofacitinib reaching at least the epidermis layer provided by a control composition replacing the ionic liquid with same amount of 2-(2-ethoxyethoxy) ethanol.

In some embodiments, the total amount of tofacitinib reaching at least the epidermis layer provided by the pharmaceutical composition is at least 2 times the total amount of tofacitinib reaching at least the epidermis layer provided by a control composition comprising a PEG-based ointment and 2% of oleyl alcohol. In some embodiments, the total amount of tofacitinib reaching at least the epidermis layer provided by the pharmaceutical composition is at least 3 times the total amount of tofacitinib reaching at least the epidermis layer provided by a control composition comprising a PEG-based ointment and 2% of oleyl alcohol. In some embodiments, the total amount of tofacitinib reaching at least the epidermis layer provided by the pharmaceutical composition is at least 4 times the total amount of tofacitinib reaching at least the epidermis layer provided by a control composition comprising a PEG-based ointment and 2% of oleyl alcohol. In some embodiments, the total amount of tofacitinib reaching at least the epidermis layer provided by the pharmaceutical composition is at least 5 times the total amount of tofacitinib reaching at least the epidermis layer provided by a control composition comprising a PEG-based ointment and 2% of oleyl alcohol.

In some embodiments, the pharmaceutical composition is administered one or more times a day. In some embodiments, the pharmaceutical composition provides reduced systemic exposure to tofacitinib as compared to therapeutically effective doses of oral tofacitinib.

In some embodiments, the fatty acid is selected from the group consisting of myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, malonic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid. In some embodiments, the fatty acid is selected from the group consisting oleic acid, geranic acid, hexanoic acid, and malonic acid. In some embodiments, the fatty acid is geranic acid.

In some embodiments, the ionic liquid is a deep eutectic solvent (DES).

In some embodiments, the ionic liquid comprises the choline cation and fatty acid anion in a molar ratio in a range of 1:1 to 1:4 of choline cation to fatty acid anion. In some embodiments, the ionic liquid comprises the choline cation and fatty acid anion in a molar ratio of 1:2 of choline cation to fatty acid anion. In some embodiments, the pharmaceutical composition consists essentially of tofacitinib and the ionic liquid. In some embodiments, the pharmaceutical composition further comprises a pharmaceutically acceptable carrier. In some embodiments, the pharmaceutically acceptable carrier is an aqueous carrier. In some embodiments, the pharmaceutically acceptable carrier comprises an ointment base.

In some embodiments, the ionic liquid is present in the pharmaceutical composition at a concentration of from about 1% to about 99%. In some embodiments, the ionic liquid is present in the pharmaceutical composition at a concentration of from about 5% to about 90%. In some embodiments, the ionic liquid is present in the pharmaceutical composition at a concentration of from about 10% to about 80%. In some embodiments, the ionic liquid is present in the pharmaceutical composition at a concentration of from about 15% to about 70%. In some embodiments, the ionic liquid is present in the pharmaceutical composition at a concentration of from about 20% to about 60%. In some embodiments, the ionic liquid is present in the pharmaceutical composition at a concentration of from about 30% to about 50%. In some embodiments, the ionic liquid is present in the pharmaceutical composition at a concentration of about 35% to about 45%. In some embodiments, the ionic liquid is present in the pharmaceutical composition at a concentration of about 40%.

In some embodiments, the pharmaceutical composition further comprises a penetration enhancer. In some embodiments, the penetration enhancer is 2-(2-ethoxyethoxy) ethanol or oleyl alcohol. In some embodiments, the penetration enhancer is 2-(2-ethoxyethoxy) ethanol. In some embodiments, the pharmaceutical composition comprises from about 1% to about 20% of2-(2-ethoxyethoxy) ethanol. In some embodiments, the pharmaceutical composition comprises from about 5% to about 15% 2-(2-ethoxyethoxy) ethanol. In some embodiments, the pharmaceutical composition comprises from about 10% 2-(2-ethoxyethoxy) ethanol.

In some embodiments, the pharmaceutical composition is essentially free of 2-(2-ethoxyethoxy) ethanol. In some embodiments, the pharmaceutical composition is essentially free of oleyl alcohol.

### Topical Gel Composition

Gels have been defined in various ways. For example, the United States Pharmacopoeia defines gels as semisolid systems consisting of either suspensions made up of small inorganic particles or large organic molecules interpenetrated by a liquid. Gels include a single-phase or a two-phase system. A single-phase gel consists of organic macromolecules distributed uniformly throughout a liquid in such a manner that no apparent boundaries exist between the dispersed macromolecules and the liquid. Some single-phase gels are prepared from synthetic macromolecules (e.g., carbomer) or from natural gums, (e.g., tragacanth). In some embodiments, single-phase gels are generally aqueous, but will also be made using alcohols and oils. Two-phase gels consist of a network of small discrete particles. The vehicle of an gel is known as the gel base

Gels can also be classified as being hydrophobic or hydrophilic. In certain embodiments, the base of a non-limiting example of a hydrophobic gel includes a liquid paraffin with polyethylene or fatty oils gelled with colloidal silica, or aluminum or zinc soaps. In contrast, the base of a non-limiting example of a hydrophilic gel includes water, glycerol, or propylene glycol gelled with a suitable gelling agent (e.g., tragacanth, starch, cellulose derivatives, carboxyvinylpolymers, and magnesium-aluminum silicates). In certain embodiments, the rheology of the compositions disclosed herein is pseudo plastic, plastic, thixotropic, or dilatant.

In some embodiments, the topical composition is a topical gel, and wherein the topically acceptable carrier comprises water and at least one viscosity-enhancing agent. In some embodiments, the viscosity-enhancing agent is selected from cellulose-based polymers, polyoxyethylene-polyoxypropylene triblock copolymers, dextran-based polymers, polyvinyl alcohol, dextrin, polyvinylpyrrolidone, polyalkylene glycols, chitosan, collagen, gelatin, hyaluronic acid, or combinations thereof.

In some embodiment, the topical gel composition described herein is a semi-solid or id in a gelled state before it is topically administered. For example, suitable viscosity-enhancing agents for such gels include by way of example only, gelling agents and suspending agents. In one embodiment, the enhanced viscosity formulation does not include a buffer. In other embodiments, the enhanced viscosity formulation includes a pharmaceutically acceptable buffer. Sodium chloride or other tonicity agents are optionally used to adjust tonicity, if necessary.

By way of example only, the topically acceptable viscosity agent includes hydroxypropyl methylcellulose, hydroxyethyl cellulose, polyvinylpyrrolidone, carboxymethyl cellulose, polyvinyl alcohol, sodium chondroitin sulfate, sodium hyaluronate. Other viscosity enhancing agents compatible with the targeted ocular site include, but are not limited to, acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose) or combinations thereof. In specific embodiments, the viscosity-enhancing excipient is a combination of MCC and CMC. In another embodiment, the viscosity-enhancing agent is a combination of carboxymethylated chitosan, or chitin, and alginate. The combination of chitin and alginate with the topical agents disclosed herein acts as a controlled release formulation, restricting the diffusion of the topical agents from the formulation. Moreover, the combination of carboxymethylated chitosan and alginate is optionally used to assist in increasing the permeability of the topical agents in the eye.

In some embodiments is an enhanced viscosity formulation, comprising from about 0.1 mM and about 100 mM of an topical agent, a pharmaceutically acceptable viscosity agent, and water for injection, the concentration of the viscosity agent in the water being sufficient to provide a enhanced viscosity formulation with a final viscosity from about 100 to about 100,000 cP. In certain embodiments, the viscosity of the gel is in the range from about 100 to about 50,000 cP, about 100 cP to about 1,000 cP, about 500 cP to about 1500 cP, about 1000 cP to about 3000 cP, about 2000 cP to about 8,000 cP, about 4,000 cP to about 50,000 cP, about 10,000 cP to about 500,000 cP, about 15,000 cP to about 1,000,000 cP. In other embodiments, when an even more viscous medium is desired, the biocompatible gel comprises at least about 35%, at least about 45%, at least about 55%, at least about 65%, at least about 70%, at least about 75%, or even at least about 80% or so by weight of the topical agent. In highly concentrated samples, the biocompatible enhanced viscosity formulation comprises at least about 25%, at least about 35%, at least about 45%, at least about 55%, at least about 65%, at least about 75%, at least about 85%, at least about 90% or at least about 95% or more by weight of the topical agent.

In one embodiment, the pharmaceutically acceptable enhanced viscosity topically acceptable formulation comprises at least one topical agent and at least one gelling agent. Suitable gelling agents for use in preparation of the gel formulation include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum and any combinations or mixtures thereof. In some other embodiments, hydroxypropylmethylcellulose (Methocel®) is utilized as the gelling agent. In certain embodiments, the viscosity enhancing agents described herein are also utilized as the gelling agent for the gel formulations presented herein.

In some embodiments, the topical gel composition described herein is an in situ gel formulation. In some instances, the in situ gel formation is based on increased pre-corneal residence time of the topical composition which improves ocular bioavailability, corneal mucoadhesion, lysosomal interaction and ionic gelation, improved corneal absorption, thermal gelation, or a combination thereof. In some instances, the in situ gel formulation is activated by pH, temperature, ion, UV, or solvent exchange.

In some instances, the topical gel composition comprises tofacitinib or pharmaceutically acceptable salt thereof and one or more gelling agents. In some instances, the gelling agent includes, but is not limited to, poloxamer (e.g. Poloxamer 407), tetronics, ethyl(hydroxyethyl) cellulose, cellulose acetate phthalate (CAP), carbopol (e.g. Carbopol 1342P NF, Carbopol 980 NF), alginates (e.g. low acetyl gellan gum (Gelrite®)), gellan, hyaluronic acid, pluronics (e.g. Pluronic F-127), chitosan, polyvinyl alcohol (PVA), polyvinylpyrrolidone (PVP), dextran, hydroxy propyl methyl cellulose (HPMC), hydroxyethylcellulose (HEC), methylcellulose (MC), thiolated xyloglucan, polymethacrilic acid (PMMA), polyethylene glycol (PEG), pseudolatexes, xyloglucans, or combinations thereof.

In some instances, the in situ gel formation further comprises a permeation enhancer. In some instances, the permeation enhancer includes surfactants (e.g. non-ionic surfactants), benzalkonium chloride, EDTA, surface-active heteroglycosides, calcium chelators, hydroxyl propyl beta cyclodextrin (HP beta CD), bile salts, and the like.

In some embodiments, other gel formulations are useful depending upon the particular topical agent, other pharmaceutical agent or excipients/additives used, and as such are considered to fall within the scope of the present disclosure. For example, other commercially-available glycerin-based gels, glycerin-derived compounds, conjugated, or crosslinked gels, matrices, hydrogels, and polymers, as well as gelatins and their derivatives, alginates, and alginate-based gels, and even various native and synthetic hydrogel and hydrogel-derived compounds are all expected to be useful in the topical agent formulations described herein. In some embodiments, topically acceptable gels include, but are not limited to, alginate hydrogels SAF®-Gel (ConvaTec, Princeton, N.J.), Duoderm® Hydroactive Gel (ConvaTec), Nu-gel® (Johnson & Johnson Medical, Arlington, Tex.); Carrasyn® (V) Acemannan Hydrogel (Carrington Laboratories, Inc., Irving, Tex.); glycerin gels Elta® Hydrogel (Swiss-American Products, Inc., Dallas, Tex.) and K-Y® Sterile (Johnson & Johnson). In further embodiments, biodegradable biocompatible gels also represent compounds present in topically acceptable formulations disclosed and described herein.

In some embodiments, the viscosity-enhancing agent is a cellulose-based polymer selected from cellulose gum, alkylcellulose, hydroxyl-alkyl cellulose, hydroxyl-alkyl alkylcellulose, carboxy-alkyl cellulose, or combinations thereof. In some embodiments, the viscosity-enhancing agent is hydroxyl-alkyl alkylcellulose. In some embodiment, the viscosity-enhancing agent is hydroxypropyl methylcellulose.

### Topical Ointment Composition

An ointment is a homogeneous, viscous, semi-solid preparation, most commonly a greasy, thick oil (e.g. oil 80%-water 20%) with a high viscosity, intended for external application to the skin or mucous membranes. Ointments have a Water number that defines the maximum amount of water that it can contain. They are used as emollients or for the application of active ingredients to the skin for protective, therapeutic, or prophylactic purposes and where a degree of occlusion is desired. Ointments are used topically on a variety of body surfaces. These include the skin and the mucous membranes of the eye (an eye ointment), vulva, anus, and nose

The vehicle of an ointment is known as the ointment base. The choice of a base depends upon the clinical indication for the ointment. The different types of ointment bases are: hydrocarbon bases, e.g. hard paraffin, soft paraffin, microcrystalline wax and ceresine; absorption bases, e.g. wool fat, beeswax; water soluble bases, e.g. macrogols 200, 300, 400; emulsifying bases, e.g. emulsifying wax, cetrimide; vegetable oils, e.g. olive oil, coconut oil, sesame oil, almond oil and peanut oil.

Ointments are formulated using hydrophobic, hydrophilic, or water-emulsifying bases to provide preparations that are immiscible, miscible, or emulsifiable with skin secretions. They can also be derived from hydrocarbon (fatty), absorption, water-removable, or water-soluble bases. The active agents are dispersed in the base, and later they get divided after the drug penetration into the target sites (e.g. membranes, skins, etc.).

The present disclosure recognizes that it is sometimes difficult to incorporate into the ointment a drug of low concentration with sufficient dose-to-dose uniformity for effectively treating a disorder or disease. In some embodiments, poly(ethylene-glycols), polyethoxylated castor oils (Cremophor®EL), alcohols having 12 to 20 carbon atoms or a mixture of two or more of said components are effective excipients for dispersing and/or dissolving effective amounts of topical drugs, in particular of ascomycins and staurosporine derivatives, in an ointment base, in particular in an ointment base substantially comprising oleaginous and hydrocarbon components, and that the resulting ointments are excellently tolerated by the skin and by ocular tissue.

The present disclosure further recognizes that topical drugs, such as tofacitinib or its pharmaceutically acceptable salts, incorporated in the ointment compositions describes herein can effectively target the choroid and/or retina in a patient when the compositions are topically administered to the ocular surface, in particular to the sclera of said patient. In some embodiments, an topical ointment composition includes an topical drug, an ointment base and an agent for dispersing and/or dissolving said drug in the ointment base, selected from a poly(ethylene-glycol), a polyethoxylated castor oil, an alcohol having 12 to 20 carbon atoms and a mixture of two or more of said components.

In some embodiments, the ointment bases include topically acceptable oil and fat bases, such as natural wax e.g. white and yellow bees wax, carnauba wax, wool wax (wool fat), purified lanolin, anhydrous lanolin; petroleum wax e.g. hard paraffin, microcrystalline wax; hydrocarbons e.g. liquid paraffin, white and yellow soft paraffin, white petrolatum, yellow petrolatum; or combinations thereof.

The above mentioned oil and fat bases are described in more detail, for instance, in the British Pharmacopoeia, Edition 2001, or the European Pharmacopoeia, 3rd Edition.

The ointment base may be present in amounts of about 50 to about 95, preferably of 70 to 90% by weight based on the total weight of the composition.

A preferred ointment base comprises a combination of one or more of one or more natural waxes like those indicated above, preferably wool wax (wool fat), and one or more hydrocarbons like those indicated above, preferably a soft paraffin or a petrolatum, more preferably in combination with liquid paraffin.

A special embodiment of the aforementioned ointment base comprises e.g. 5 to 17 parts by weight of wool fat, and 50 to 65 parts by weight of white petrolatum as well as 20 to 30 parts by weight of liquid paraffin.

The agent for dispersing and/or dissolving the topical drug in the ointment base may be selected from a poly(ethylene-glycol), a polyethoxylated castor oil, an alcohol having 12 to 20 carbon atoms and a mixture of two or more of said components. The agent is preferably used in amounts of 1 to 20 percent, more preferably 1 to 10 percent by weight of the entire semisolid topical composition.

Alcohols having 12 to 20 carbon atoms include particularly stearyl alcohol (C18H37OH), cetyl alcohol (C16H33OH) and mixtures thereof. Preferred are so-called cetostearyl alcohols, mixtures of solid alcohols substantially consisting of stearyl and cetyl alcohol and preferably comprising not less than 40 percent by weight of stearyl alcohol and a sum of stearyl alcohol and cetyl alcohol amounting to at least 90 percent by weight, and compositions comprising not less than 80 percent by weight of cetylstearyl alcohol and an emulsifier, in particular sodium cetostearyl sulfate and/or sodium lauryl sulfate, preferably in amounts not less than 7 percent by weight of emulsifier.

Polyethoxylated castor oils are reaction products of natural or hydrogenated castor oils and ethylene glycol. Such products may be obtained in known manner, e.g. by reaction of a natural or hydrogenated castor oil or fractions thereof with ethylene oxide, e.g. in a molar ratio of from about 1:30 to about 1:60, with optional removal of free polyethylene glycol components from the product, e.g. in accordance with the methods disclosed in German Auslegeschriften 1,182,388 and 1,518,819. Especially suitable and preferred is a product commercially available under the trade name Cremophor®EL having a molecular weight (by steam osmometry)=ca. 1630, a saponification no.=ca. 65-70, an acid no.=ca. 2, an iodine no.=ca. 28-32 and an nD 25=ca.1.471. Also suitable for use in this category is, for instance, Nikkol®HCO-60, a reaction product of hydrogenated castor oil and ethylene oxide exhibiting the following characteristics: acid no.=ca. 0.3; saponification no.=ca. 47.4; hydroxy value=ca. 42.5. pH (5%)=ca. 4.6; Color APHA=ca. 40; m.p.=ca. 36.0° C.; Freezing point=ca. 32.4° C.; H₂O content (%, KF)=ca. 0.03.

Poly(ethylene-glycols) are used in some embodiments as the agent for dispersing and/or dissolving the topical drug in the ointment base according to the present disclosure. Suitable poly(ethylene-glycol) s are typically mixtures of polymeric compounds of the general formula H—(OCH2-CH2)nOH, wherein the index n may typically range from 4 to 230 and the mean molecular weight from about 200 to about 10000. Preferably n is a number from about 6 to about 22 and the mean molecular weight between about 300 and about 1000, more preferably n ranges from about 6 to about 13 and the mean molecular weight from about 300 to about 600, most preferably n has a value of about 8.5 to about 9 and the relative molecular weight is about 400. Suitable poly(ethylene-glycols) are readily available commercially, for example poly(ethylene-glycols) having a mean molecular weight of about 200, 300, 400, 600, 1000, 1500, 2000, 3000, 4000, 6000, 8000 and 10000.

The poly(ethylene-glycols), in particular the preferred types described in the foregoing paragraph, are preferably used in amounts of 1 to 10, more preferably 1 to 5 percent by weight of the entire semisolid topical composition.

An especially preferred embodiment of the compositions according to the instant disclosure comprises an agent for dispersing and/or dissolving of the drug in the ointment base which is selected from a poly(ethylene-glycol), a polyethoxylated castor oil and preferably a mixture of said components.

### Gel/Ointment Viscosity

In some embodiments, the composition has a Brookfield RVDV viscosity of from about 10,000 to about 300,000 cps at about 20° C. and sheer rate of 1 s⁻¹. In some embodiments, the composition has a Brookfield RVDV viscosity of from about 15,000 to about 200,000 cps at about 20° C. and sheer rate of 1 s⁻¹. In some embodiments, the composition has a Brookfield RVDV viscosity of from about 50,000 to about 150,000 cps at about 20° C. and sheer rate of 1 s⁻¹. In some embodiments, the composition has a Brookfield RVDV viscosity of from about 70,000 to about 130,000 cps at about 20° C. and sheer rate of 1 s⁻¹. In some embodiments, the composition has a Brookfield RVDV viscosity of from about 90,000 to about 110,000 cps at about 20° C. and sheer rate of 1 s⁻¹.

In some embodiments, the topical gel formulation contains a viscosity enhancing agent sufficient to provide a viscosity of between about 500 and 1,000,000 centipoise, between about 750 and 1,000,000 centipoise; between about 1000 and 1,000,000 centipoise; between about 1000 and 400,000 centipoise; between about 2000 and 100,000 centipoise; between about 3000 and 50,000 centipoise; between about 4000 and 25,000 centipoise; between about 5000 and 20,000 centipoise; or between about 6000 and 15,000 centipoise. In some embodiments, the topical gel formulation contains a viscosity enhancing agent sufficient to provide a viscosity of between about 50,0000 and 1,000,000 centipoise.

In some embodiments, the compositions described herein are low viscosity compositions at body temperature. In some embodiments, low viscosity compositions contain from about 1% to about 10% of a viscosity enhancing agent (e.g., gelling components such as polyoxyethylene-polyoxypropylene copolymers). In some embodiments, low viscosity compositions contain from about 2% to about 10% of a viscosity enhancing agent (e.g., gelling components such as polyoxyethylene-polyoxypropylene copolymers). In some embodiments, low viscosity compositions contain from about 5% to about 10% of a viscosity enhancing agent (e.g., gelling components such as polyoxyethylene-polyoxypropylene copolymers). In some embodiments, low viscosity compositions are substantially free of a viscosity enhancing agent (e.g., gelling components such as polyoxyethylene-polyoxypropylene copolymers). In some embodiments, a low viscosity topical agent composition described herein provides an apparent viscosity of from about 100 cP to about 10,000 cP. In some embodiments, a low viscosity topical agent composition described herein provides an apparent viscosity of from about 500 cP to about 10,000 cP. In some embodiments, a low viscosity topical agent composition described herein provides an apparent viscosity of from about 1000 cP to about 10,000 cP.

In some embodiments, the compositions described herein are viscous compositions at body temperature. In some embodiments, viscous compositions contain from about 10% to about 25% of a viscosity enhancing agent (e.g., gelling components such as polyoxyethylene-polyoxypropylene copolymers). In some embodiments, the viscous compositions contain from about 14% to about 22% of a viscosity enhancing agent (e.g., gelling components such as polyoxyethylene-polyoxypropylene copolymers). In some embodiments, the viscous compositions contain from about 15% to about 21% of a viscosity enhancing agent (e.g., gelling components such as polyoxyethylene-polyoxypropylene copolymers). In some embodiments, a viscous topical composition described herein provides an apparent viscosity of from about 100,000 cP to about 1,000,000 cP. In some embodiments, a viscous topical composition described herein provides an apparent viscosity of from about 150,000 cP to about 500,000 cP. In some embodiments, a viscous topical composition described herein provides an apparent viscosity of from about 250,000 cP to about 500,000 cP. In some of such embodiments, a viscous topical composition is a liquid at room temperature and gels at about between room temperature and body temperature (including an individual with a serious fever, e.g., up to about 42° C.). In some embodiments, a viscous topical composition is administered as monotherapy for treatment of an topical disease or condition described herein.

In some embodiments, the viscosity of the gel formulations presented herein is measured by any means described. For example, in some embodiments, an LVDV-II+CP Cone Plate Viscometer and a Cone Spindle CPE-40 is used to calculate the viscosity of the gel formulation described herein. In other embodiments, a Brookfield (spindle and cup) viscometer is used to calculate the viscosity of the gel formulation described herein. In some embodiments, the viscosity ranges referred to herein are measured at room temperature. In other embodiments, the viscosity ranges referred to herein are measured at body temperature (e.g., at the average body temperature of a healthy human).

### Gel/Ointment Dose-To-Dose Uniformity

Typical topical gels are packaged in eye drop bottles and administered as drops. For example, a single administration (i.e. a single dose) of an topical gel may include a single drop, two drops, three drops or more into the eyes of the patient. Furthermore, typical topical ointments are packaged in tubes or other squeezable containers with a dispensing nozzle through which strips of the ointment are delivered. For example, a single administration (i.e. a single dose) of an topical ointment may include a single strip, or multiple strips into the eyes of the patient. In some embodiments, one dose of the topical gel described herein is one drop of the gel composition from the eye drop bottle. In some embodiments, one dose of the topical ointment is one strip of the ointment composition dispensed through the nozzle of a dispersing tube.

In some cases, described herein include topical gel compositions which provide a dose-to-dose uniform concentrations. In some instances, the dose-to-dose uniform concentration does not present significant variations of drug content from one dose to another. In some instances, the dose-to-dose uniform concentration does provide consistant drug content from one dose to another.

In some cases, described herein include topical ointment compositions which provide a dose-to-dose uniform concentrations. In some instances, the dose-to-dose uniform concentration does not present significant variations of drug content from one dose to another. In some instances, the dose-to-dose uniform concentration does provide consistant drug content from one dose to another.

In some embodiments, the composition has a dose-to-dose topical agent concentration variation of less than 50%. In some embodiments, the composition has a dose-to-dose topical agent concentration variation of less than 40%. In some embodiments, the composition has a dose-to-dose topical agent concentration variation of less than 30%. In some embodiments, the composition has a dose-to-dose topical agent concentration variation of less than 20%. In some embodiments, the composition has a dose-to-dose topical agent concentration variation of less than 10%. In some embodiments, the composition has a dose-to-dose topical agent concentration variation of less than 5%.

In some embodiments, the dose-to-dose topical agent concentration variation is based on 10 consecutive doses. In some embodiments, the dose-to-dose topical agent concentration variation is based on 8 consecutive doses. In some embodiments, the dose-to-dose topical agent concentration variation is based on 5 consecutive doses. In some embodiments, the dose-to-dose topical agent concentration variation is based on 3 consecutive doses. In some embodiments, the dose-to-dose topical agent concentration variation is based on 2 consecutive doses.

A nonsettling formulation should not require shaking to disperse drug uniformly. A “no-shake” formulation is potentially advantageous over formulations that require shaking for the simple reason that patients' shaking behavior is a major source of variability in the amount of drug dosed. It has been reported that patients often times do not or forget to shake their topical compositions that requires shaking before administering a dose, despite the instructions to shake that were clearly marked on the label. On the other hand, even for those patients who do shake the product, it is normally not possible to determine whether the shaking is adequate in intensity and/or duration to render the product uniform. In some embodiments, the topical gel compositions and topical ointment compositions described herein are “no-shake” formulations that maintained the dose-to-dose uniformity described herein.

To evaluate the dose-to-dose uniformity, drop bottles or tubes containing the topical aqueous compositions, the topical gel compositions, or topical ointment compositions are stored upright for a minimum of 12 hours prior to the start of the test. To simulate the recommended dosing of these products, predetermined number of drops or strips are dispensed from each commercial bottles or tubes at predetermined time intervals for an extended period of time or until no product was left in the bottle or tube. All drops and strips are dispensed into tared glass vials, capped, and stored at room temperature until analysis. Concentrations of tofacitinib or pharmaceutically acceptable salt thereof in the expressed drops were determined using a reverse-phase HPLC method.

### Topical Composition Skin Compatibility

In some instances, the composition described herein further comprises a tonicity adjusting agent. Tonicity adjusting agent is an agent introduced into a preparation such as an topical composition to reduce local irritation by preventing osmotic shock at the site of application. In some instances, buffer solution and/or a pH adjusting agent that broadly maintains the topical solution at a particular ion concentration and pH are considered as tonicity adjusting agents. In some cases, tonicity adjusting agents include various salts, such as halide salts of a monovalent cation. In some cases, tonicity adjusting agents include mannitol, sorbitol, dextrose, sucrose, urea, and glycerin. In some instances, suitable tonicity adjustors comprise sodium chloride, sodium nitrate, sodium sulfate, sodium bisulfate, potassium chloride, calcium chloride, magnesium chloride, zinc chloride, potassium acetate, sodium acetate, sodium bicarbonate, sodium carbonate, sodium thiosulfate, magnesium sulfate, disodium hydrogen phosphate, sodium dihydrogen phosphate, potassium dihydrogen phosphate, dextrose, mannitol, sorbitol, dextrose, sucrose, urea, propylene glycol, glycerin, or a combination thereof.

In some cases, the composition described herein further comprises a pH adjusting agent. The pH adjusting agent is used which can be acid or base. The base can be oxides, hydroxides, carbonates, bicarbonates and the likes. The oxides can be metal oxides such as calcium oxide, magnesium oxide and the likes; hydroxides can be of alkali metals and alkaline earth metals such as sodium hydroxide, potassium hydroxide, calcium hydroxide and the likes and carbonates can be sodium carbonate, sodium bicarbonates, potassium bicarbonates and the likes. The acid can be mineral acid and organic acids such as hydrochloric acid, nitric acid, phosphoric acid, acetic acid, citric acid, fumaric acid, malic acid tartaric acid and the likes. In some instances, the pH adjusting agent includes, but is not limited to, acetate, bicarbonate, ammonium chloride, citrate, phosphate, pharmaceutically acceptable salts thereof and combinations or mixtures thereof.

As described elsewhere herein, the pH of the composition is between about 4.5 and about 7.5. In some instances, the pH of the composition described herein is less than about 7. In some instances, the pH of the composition described herein is less than about 6.8. In some instances, the pH of the composition described herein is less than about 6.5. In some instances, the pH of the composition described herein is less than about 6.3. In some instances, the pH of the composition described herein is less than about 6.1. In some instances, the pH of the composition described herein is less than about 6. In some instances, the pH of the composition described herein is less than about 5.9. In some instances, the pH of the composition described herein is less than about 5.8.

In some embodiments, the pH of the composition described herein is associated with the stability of the composition. In some embodiments, a stable composition comprises a pH of between about 4.5 and about 7.5. In some embodiments, a stable composition comprises a pH of less than about 7. In some embodiments, a stable composition comprises a pH of less than about 6.8. In some embodiments, a stable composition comprises a pH of less than about 6.5. In some embodiments, a stable composition comprises a pH of less than about 6.3. In some embodiments, a stable composition comprises a pH of less than about 6.1. In some embodiments, a stable composition comprises a pH of less than about 6. In some embodiments, a stable composition comprises a pH of less than about 5.9. In some embodiments, a stable composition comprises a pH of less than about 5.8.

In some embodiments, the composition described herein comprises a buffer. In some embodiments, a buffer is selected from borates, borate-polyol complexes, phosphate buffering agents, citrate buffering agents, acetate buffering agents, carbonate buffering agents, organic buffering agents, amino acid buffering agents, or combinations thereof.

In some instances, borates include boric acid, salts of boric acid, other pharmaceutically acceptable borates, and combinations thereof. In some cases, borates include boric acid, sodium borate, potassium borate, calcium borate, magnesium borate, manganese borate, and other such borate salts.

As used herein, the term polyol includes any compound having at least one hydroxyl group on each of two adjacent carbon atoms that are not in trans configuration relative to each other. The polyols can be linear or cyclic, substituted or unsubstituted, or mixtures thereof, so long as the resultant complex is water soluble and pharmaceutically acceptable. In some instances, examples of polyol include: sugars, sugar alcohols, sugar acids and uronic acids. In some cases, polyols include, but are not limited to: mannitol, glycerin, xylitol and sorbitol.

In some embodiments, phosphate buffering agents include phosphoric acid; alkali metal phosphates such as disodium hydrogen phosphate, sodium dihydrogen phosphate, trisodium phosphate, dipotassium hydrogen phosphate, potassium dihydrogen phosphate, and tripotassium phosphate; alkaline earth metal phosphates such as calcium phosphate, calcium hydrogen phosphate, calcium dihydrogen phosphate, monomagnesium phosphate, dimagnesium phosphate (magnesium hydrogen phosphate), and trimagnesium phosphate; ammonium phosphates such as diammonium hydrogen phosphate and ammonium dihydrogen phosphate; or a combination thereof. In some instances, the phosphate buffering agent is an anhydride. In some instances, the phosphate buffering agent is a hydrate.

In some embodiments, borate-polyol complexes include those described in U.S. Pat. No. 6,503,497. In some instances, the borate-polyol complexes comprise borates in an amount of from about 0.01 to about 2.0% w/v, and one or more polyols in an amount of from about 0.01% to about 5.0% w/v.

In some cases, citrate buffering agents include citric acid and sodium citrate.

In some instances, acetate buffering agents include acetic acid, potassium acetate, and sodium acetate.

In some instances, carbonate buffering agents include sodium bicarbonate and sodium carbonate.

In some cases, organic buffering agents include Good's Buffer, such as for example 2-(N-morpholino) ethanesulfonic acid (MES), N-(2-Acetamido)iminodiacetic acid, N-(Carbamoylmethyl)iminodiacetic acid (ADA), piperazine-N,N′-bis(2-ethanesulfonic acid (PIPES), N-(2-acetamido)-2-aminoethanesulfonic acid (ACES), β-Hydroxy-4-morpholinepropanesulfonic acid, 3-Morpholino-2-hydroxypropanesulfonic acid (MOPSO), cholamine chloride, 3-(N-morpholino)propansulfonic acid (MOPS), N,N-bis(2-hydroxyethyl)-2-aminoethanesulfonic acid (BES), 2-[(2-Hydroxy-1,1-bis(hydroxymethyl)ethyl)amino]ethanesulfonic acid (TES), 4-(2-hydroxyethyl)-1-piperazineethanesulfonic acid (HEPES), 3-(N,N-Bis[2-hydroxyethyl]amino)-2-hydroxypropanesulfonic acid (DIPSO), acetamidoglycine, 3-{[1,3-Dihydroxy-2-(hydroxymethyl)-2-propanyl]amino}-2-hydroxy-1-propanesulfonic acid (TAPSO), piperazine-1,4,-bis(2-hydroxypropanesulphonic acid) (POPSO), 4-(2-hydroxyethyl) piperazine-1-(2-hydroxypropanesulfonic acid) hydrate (HEPPSO), 3-[4-(2-hydroxyethyl)-1-piperazinyl]propanesulfonic acid (HEPPS), tricine, glycinamide, bicine or N-tris(hydroxymethyl)methyl-3-aminopropanesulfonic acid sodium (TAPS); glycine; and diethanolamine (DEA).

In some cases, amino acid buffering agents include taurine, aspartic acid and its salts (e.g., potassium salts, etc), E-aminocaproic acid, and the like.

In some instances, the composition described herein further comprises a disinfecting agent. In some cases, disinfecting agents include polymeric biguanides, polymeric quaternary ammonium compounds, chlorites, bisbiguanides, chlorite compounds (e.g. potassium chlorite, sodium chlorite, calcium chlorite, magnesium chlorite, or mixtures thereof), and a combination thereof.

In some instances, the composition described herein further comprises a preservative. In some cases, a preservative is added at a concentration to a composition described herein to prevent the growth of or to destroy a microorganism introduced into the composition. In some instances, microorganisms refer to bacteria (e.g. Proteus mirabilis, Serratia marcesens), virus (e.g. Herpes simplex virus, herpes zoster virus), fungus (e.g. fungi from the genus Fusarium), yeast (e.g. Candida albicans), parasites (e.g. Plasmodium spp., Gnathostoma spp.), protozoan (e.g. Giardia lamblia), nematodes (e.g. Onchocercus volvulus), worm (e.g. Dirofilaria immitis), and/or amoeba (e.g. Acanthameoba).

In some instances, the concentration of the preservative is between about 0.0001% and about 1%, about 0.001% and about 0.8%, about 0.004% and about 0.5%, about 0.008% and about 0.1%, and about 0.01% and about 0.08%. In some cases, the concentration of the preservatives is about 0.001%, 0.002%, 0.003%, 0.004%, 0.005%, 0.006%, 0.008%, 0.009%, 0.009%, 0.01%, 0.015%, 0.02%, 0.025%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9% or 1.0%.

In some embodiments, the preservative is selected from benzalkonium chloride, cetrimonium, sodium perborate, stabilized oxychloro complex, SofZia (Alcon), polyquaternium-1, chlorobutanol, edetate disodium, and polyhexamethylene biguanide.

In some instances, the composition described herein further comprises a stabilizer. Stabilizers that are useful in the topically acceptable formulations disclosed herein include, for example, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, and combinations thereof. In some embodiments, amide analogues of stabilizers are also used. In further embodiments, the chosen stabilizer changes the hydrophobicity of the formulation, improves the mixing of various components in the formulation, controls the moisture level in the formula, or controls the mobility of the phase.

In other embodiments, stabilizers are present in sufficient amounts to inhibit the degradation of the topical agent. Examples of such stabilizing agents, include, but are not limited to: glycerol, methionine, monothioglycerol, EDTA, ascorbic acid, polysorbate 80, polysorbate 20, arginine, heparin, dextran sulfate, cyclodextrins, pentosan polysulfate and other heparinoids, divalent cations such as magnesium and zinc, or combinations thereof.

Additional useful stabilization agents for topically acceptable formulations include one or more anti-aggregation additives to enhance stability of topical formulations by reducing the rate of protein aggregation. The anti-aggregation additive selected depends upon the nature of the conditions to which the topical agents, for example tofacitinib or pharmaceutically acceptable salt thereof, are exposed. For example, certain formulations undergoing agitation and thermal stress require a different anti-aggregation additive than a formulation undergoing lyophilization and reconstitution. Useful anti-aggregation additives include, by way of example only, urea, guanidinium chloride, simple amino acids such as glycine or arginine, sugars, polyalcohols, polysorbates, polymers such as polyethylene glycol and dextrans, alkyl saccharides, such as alkyl glycoside, and surfactants.

Other useful formulations optionally include one or more topically acceptable antioxidants to enhance chemical stability where required. Suitable antioxidants include, by way of example only, ascorbic acid, methionine, sodium thiosulfate and sodium metabisulfite. In one embodiment, antioxidants are selected from metal chelating agents, thiol containing compounds and other general stabilizing agents.

Still other useful compositions include one or more topically acceptable surfactants to enhance physical stability or for other purposes. Suitable nonionic surfactants include, but are not limited to, polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

In other embodiments, an additional surfactant (co-surfactant) and/or buffering agent is combined with one or more of the pharmaceutically acceptable vehicles previously described herein so that the surfactant and/or buffering agent maintains the product at an optimal pH for stability. Suitable co-surfactants include, but are not limited to: a) natural and synthetic lipophilic agents, e.g., phospholipids, cholesterol, and cholesterol fatty acid esters and derivatives thereof; b) nonionic surfactants, which include for example, polyoxyethylene fatty alcohol esters, sorbitan fatty acid esters (Spans), polyoxyethylene sorbitan fatty acid esters (e.g., polyoxyethylene (20) sorbitan monooleate (Tween 80), polyoxyethylene (20) sorbitan monostearate (Tween 60), polyoxyethylene (20) sorbitan monolaurate (Tween 20) and other Tweens, sorbitan esters, glycerol esters, e.g., Myrj and glycerol triacetate (triacetin), polyethylene glycols, cetyl alcohol, cetostearyl alcohol, stearyl alcohol, polysorbate 80, poloxamers, poloxamines, polyoxyethylene castor oil derivatives (e.g., Cremophor® RH40, Cremphor A25, Cremphor A20, Cremophor® EL) and other Cremophors, sulfosuccinates, alkyl sulphates (SLS); PEG glyceryl fatty acid esters such as PEG-8 glyceryl caprylate/caprate (Labrasol), PEG-4 glyceryl caprylate/caprate (Labrafac Hydro WL 1219), PEG-32 glyceryl laurate (Gelucire 444/14), PEG-6 glyceryl mono oleate (Labrafil M 1944 CS), PEG-6 glyceryl linoleate (Labrafil M 2125 CS); propylene glycol mono- and di-fatty acid esters, such as propylene glycol laurate, propylene glycol caprylate/caprate; Brij® 700, ascorbyl-6-palmitate, stearylamine, sodium lauryl sulfate, polyoxethyleneglycerol triiricinoleate, and any combinations or mixtures thereof; c) anionic surfactants include, but are not limited to, calcium carboxymethylcellulose, sodium carboxymethylcellulose, sodium sulfosuccinate, dioctyl, sodium alginate, alkyl polyoxyethylene sulfates, sodium lauryl sulfate, triethanolamine stearate, potassium laurate, bile salts, and any combinations or mixtures thereof; and d) cationic surfactants such as cetyltrimethylammonium bromide, and lauryldimethylbenzyl-ammonium chloride.

In a further embodiment, when one or more co-surfactants are utilized in the topically acceptable formulations of the present disclosure, they are combined, e.g., with a pharmaceutically acceptable vehicle and is present in the final formulation, e.g., in an amount ranging from about 0.1% to about 20%, from about 0.5% to about 10%.

In one embodiment, the surfactant has an HLB value of 0 to 20. In additional embodiments, the surfactant has an HLB value of 0 to 3, of 4 to 6, of 7 to 9, of 8 to 18, of 13 to 15, of 10 to 18.

### Skin Penetration and Systemic Exposure

Disclosed herein, in certain embodiments, is a method of locally inhibiting activity of at least one of JAK-1, JAK-2, TYK-2, and JAK-3 within an epidermis layer, a dermis layer, a subcutaneous tissue layer, or a muscle tissue, the method comprising administering to the skin a pharmaceutical composition comprising tofacitinib or a pharmaceutically acceptable salt thereof, and an ionic liquid in an amount sufficient to allow a therapeutically effective amount of tofacitinib to reach a target depth within or beyond the skin, wherein the ionic liquid comprises a choline cation and a fatty acid anion.

In some embodiments, the pharmaceutical composition comprises about 0.01% to about 10% of tofacitinib or a pharmaceutically acceptable salt thereof. In some embodiments, the pharmaceutical composition comprises about 0.1% to about 8% of tofacitinib or a pharmaceutically acceptable salt thereof. In some embodiments, the topical ointment comprises about 0.1% to about 5% of tofacitinib or a pharmaceutically acceptable salt thereof.

In some embodiments, the target depth is from about 0.05 mm to about 20 mm. In some embodiments, the target depth is from about 0.1 mm to about 15 mm. In some embodiments, the target depth is from about 1 mm to about 10 mm.

In some embodiments, the pharmaceutical composition allows therapeutically effective amount of tofacitinib to reach at least the epidermis layer. In some embodiments, the amount of tofacitinib at the epidermis layer is sufficient to inhibit activity of at least one of JAK-1, JAK-2, TYK-2, and JAK-3 within the epidermis layer.

In some embodiments, the pharmaceutical composition allows therapeutically effective amount of tofacitinib to reach at least the dermis layer. In some embodiments, the amount of tofacitinib at the dermis layer is sufficient to inhibit activity of at least one of JAK-1, JAK-2, TYK-2, and JAK-3 within the dermis layer.

In some embodiments, the pharmaceutical composition allows therapeutically effective amount of tofacitinib to reach at least the the subcutaneous tissue layer. In some embodiments, the amount of tofacitinib at the subcutaneous tissue layer is sufficient to inhibit activity of at least one of JAK-1, JAK-2, TYK-2, and JAK-3 within the subcutaneous tissue layer.

In some embodiments, the pharmaceutical composition allows therapeutically effective amount of tofacitinib to reach at least the muscle tissue. In some embodiments, the amount of tofacitinib at the muscle tissue is sufficient to inhibit activity of at least one of JAK-1, JAK-2, TYK-2, and JAK-3 within the muscle tissue.

In some embodiments, the total amount of tofacitinib reaching at least the epidermis layer provided by the pharmaceutical composition is at least 1.5 times the total amount of tofacitinib reaching at least the epidermis layer provided by a control composition replacing the ionic liquid with same amount of 2-(2-ethoxyethoxy) ethanol. In some embodiments, the total amount of tofacitinib reaching at least the epidermis layer provided by the pharmaceutical composition is at least 2 times the total amount of tofacitinib reaching at least the epidermis layer provided by a control composition replacing the ionic liquid with same amount of 2-(2-ethoxyethoxy) ethanol. In some embodiments, the total amount of tofacitinib reaching at least the epidermis layer provided by the pharmaceutical composition is at least 2.5 times the total amount of tofacitinib reaching at least the epidermis layer provided by a control composition replacing the ionic liquid with same amount of 2-(2-ethoxyethoxy) ethanol. In some embodiments, the total amount of tofacitinib reaching at least the epidermis layer provided by the pharmaceutical composition is at least 3 times the total amount of tofacitinib reaching at least the epidermis layer provided by a control composition replacing the ionic liquid with same amount of 2-(2-ethoxyethoxy) ethanol.

In some embodiments, the total amount of tofacitinib reaching at least the epidermis layer provided by the pharmaceutical composition is at least 2 times the total amount of tofacitinib reaching at least the epidermis layer provided by a control composition comprising a PEG-based ointment and 2% of oleyl alcohol. In some embodiments, the total amount of tofacitinib reaching at least the epidermis layer provided by the pharmaceutical composition is at least 3 times the total amount of tofacitinib reaching at least the epidermis layer provided by a control composition comprising a PEG-based ointment and 2% of oleyl alcohol. In some embodiments, the total amount of tofacitinib reaching at least the epidermis layer provided by the pharmaceutical composition is at least 4 times the total amount of tofacitinib reaching at least the epidermis layer provided by a control composition comprising a PEG-based ointment and 2% of oleyl alcohol. In some embodiments, the total amount of tofacitinib reaching at least the epidermis layer provided by the pharmaceutical composition is at least 5 times the total amount of tofacitinib reaching at least the epidermis layer provided by a control composition comprising a PEG-based ointment and 2% of oleyl alcohol.

In some embodiments, the pharmaceutical composition is administered one or more times a day. In some embodiments, the pharmaceutical composition provides reduced systemic exposure to tofacitinib as compared to therapeutically effective doses of oral tofacitinib.

In some embodiments, the fatty acid is selected from the group consisting of myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, malonic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid. In some embodiments, the fatty acid is selected from the group consisting oleic acid, geranic acid, hexanoic acid, and malonic acid. In some embodiments, the fatty acid is geranic acid.

In some embodiments, the ionic liquid is a deep eutectic solvent (DES). In some embodiments, the ionic liquid comprises the choline cation and fatty acid anion in a molar ratio in a range of 1:1 to 1:4 of choline cation to fatty acid anion. In some embodiments, the ionic liquid comprises the choline cation and fatty acid anion in a molar ratio of 1:2 of choline cation to fatty acid anion.

In some embodiments, the pharmaceutical composition consists essentially of tofacitinib and the ionic liquid. In some embodiments, the pharmaceutical composition further comprises a pharmaceutically acceptable carrier. In some embodiments, the pharmaceutically acceptable carrier is an aqueous carrier. In some embodiments, the pharmaceutically acceptable carrier comprises an ointment base.

In some embodiments, the ionic liquid is present in the pharmaceutical composition at a concentration of from about 1% to about 99%. In some embodiments, the ionic liquid is present in the pharmaceutical composition at a concentration of from about 5% to about 90%. In some embodiments, the ionic liquid is present in the pharmaceutical composition at a concentration of from about 10% to about 80%. In some embodiments, the ionic liquid is present in the pharmaceutical composition at a concentration of from about 15% to about 70%. In some embodiments, the ionic liquid is present in the pharmaceutical composition at a concentration of from about 20% to about 60%. In some embodiments, the ionic liquid is present in the pharmaceutical composition at a concentration of from about 30% to about 50%. In some embodiments, the ionic liquid is present in the pharmaceutical composition at a concentration of about 35% to about 45. In some embodiments, the ionic liquid is present in the pharmaceutical composition at a concentration of about 40%.

In some embodiments, the pharmaceutical composition further comprises a penetration enhancer. In some embodiments, the penetration enhancer is 2-(2-ethoxyethoxy) ethanol or oleyl alcohol. In some embodiments, the penetration enhancer is 2-(2-ethoxyethoxy) ethanol. In some embodiments, the pharmaceutical composition comprises from about 1% to about 20% of 2-(2-ethoxyethoxy) ethanol. In some embodiments, the pharmaceutical composition comprises from about 5% to about 15% 2-(2-ethoxyethoxy) ethanol. In some embodiments, the pharmaceutical composition comprises from about 10% 2-(2-ethoxyethoxy) ethanol.

In some embodiments, the pharmaceutical composition is essentially free of 2-(2-ethoxyethoxy) ethanol. In some embodiments, the pharmaceutical composition is essentially free of oleyl alcohol.

In some embodiments, the amount of the composition administered to the individual and the length of treatment depends on the attributes of the individual including, but not limited to, state of health, weight, severity of the condition, previous therapy, and judgement of the treating physician. In some embodiments, the amount of the composition administered to the individual is determined by routine experimentation (e.g., a dose escalation clinical trial).

In some embodiments, topical compositions as described herein inhibits activity of at least one of JAK-1, JAK-2, TYK-2, and JAK-3 within the epidermis layer by 10%, 15%, 20%, 25%, 30%, 35%, 40%, 45%, 50%, 55%, 60%, 65%, 70%, 75%, 80%, 85%, 90%, 95%, or more than 95%.

In some embodiments, topical compositions as described herein inhibits activity of at least one of JAK-1, JAK-2, TYK-2, and JAK-3 within the dermis layer by 10%, 15%, 20%, 25%, 30%, 35%, 40%, 45%, 50%, 55%, 60%, 65%, 70%, 75%, 80%, 85%, 90%, 95%, or more than 95%.

In some embodiments, topical compositions as described herein inhibits activity of at least one of JAK-1, JAK-2, TYK-2, and JAK-3 within the subcutaneous tissue layer by 10%, 15%, 20%, 25%, 30%, 35%, 40%, 45%, 50%, 55%, 60%, 65%, 70%, 75%, 80%, 85%, 90%, 95%, or more than 95%.

In some embodiments, topical compositions as described herein inhibits activity of at least one of JAK-1, JAK-2, TYK-2, and JAK-3 within the muscle by 10%, 15%, 20%, 25%, 30%, 35%, 40%, 45%, 50%, 55%, 60%, 65%, 70%, 75%, 80%, 85%, 90%, 95%, or more than 95%.

In some embodiments, the composition is applied to the skin of the individual once a day. In some embodiments, the composition is applied to the skin of the individual 1, 2, 3, 4, or 5 times a day. In some embodiments, the composition is applied to the skin of the individual 2 times a day. In some embodiments, the composition is applied to the skin of the individual 2 times a day, e.g., morning and evening. In some embodiments, the composition is applied to the skin of the individual every day, every other day, every three days, twice a week, once a week, or once a month. In some embodiments, the composition is applied to the skin of the individual once. In some embodiments, the composition is applied to the skin of the individual for a period of time of 1 week, 2 weeks, 3 weeks, 1 month, 2 months, or 3 months or more. In some embodiments, the composition is applied to the skin until the symptoms of the disease or condition associated with activity of JAK(s), such as JAK-1, JAK-2, TYK-2, and/or JAK-3, are eliminated. In some embodiments, the composition is applied to the skin until the symptoms of the disease or condition associated with activity of JAK(s), such as JAK-1, JAK-2, TYK-2, and/or JAK-3, are reduced.

### Increased Dermal Bioavailability of Tofacitinib by Topical Administration

In some embodiments, the administration by topical route increases the dermal bioavailability of the tofacitinib by about 1-fold to about 1000-fold compared to the dermal bioavailability of the tofacitinib of a comparative composition administered by oral route at the same delivery dose. In some embodiments, the administration by topical route increases the dermal bioavailability of the tofacitinib by about 1-fold to about 500-fold compared to the dermal bioavailability of the tofacitinib of a comparative composition administered by oral route at the same delivery dose. In some embodiments, the administration by topical route increases the dermal bioavailability of the tofacitinib by about 1-fold to about 100-fold compared to the dermal bioavailability of the tofacitinib of a comparative composition administered by oral route at the same delivery dose. In some embodiments, the administration by topical route increases the dermal bioavailability of the tofacitinib by about 1-fold to about 50-fold compared to the dermal bioavailability of the tofacitinib of a comparative composition administered by oral route at the same delivery dose. In some embodiments, the administration by topical route increases the dermal bioavailability of the tofacitinib by about 1-fold to about 20-fold compared to the dermal bioavailability of the tofacitinib of a comparative composition administered by oral route at the same delivery dose. In some embodiments, the administration by topical route increases the dermal bioavailability of the tofacitinib by about 1-fold to about 10-fold compared to the dermal bioavailability of the tofacitinib of a comparative composition administered by oral route at the same delivery dose.

In some embodiments, the administration by topical route increases the dermal bioavailability of the tofacitinib by about 1-fold compared to the dermal bioavailability of the tofacitinib of a comparative composition administered by oral route at the same delivery dose. In some embodiments, the administration by topical route increases the dermal bioavailability of the tofacitinib by about 2-fold compared to the dermal bioavailability of the tofacitinib of a comparative composition administered by oral route at the same delivery dose. In some embodiments, the administration by topical route increases the dermal bioavailability of the tofacitinib by about 3-fold compared to the dermal bioavailability of the tofacitinib of a comparative composition administered by oral route at the same delivery dose. In some embodiments, the administration by topical route increases the dermal bioavailability of the tofacitinib by about 4-fold compared to the dermal bioavailability of the tofacitinib of a comparative composition administered by oral route at the same delivery dose. In some embodiments, the administration by topical route increases the dermal bioavailability of the tofacitinib by about 5-fold compared to the dermal bioavailability of the tofacitinib of a comparative composition administered by oral route at the same delivery dose. In some embodiments, the administration by topical route increases the dermal bioavailability of the tofacitinib by about 10-fold compared to the dermal bioavailability of the tofacitinib of a comparative composition administered by oral route at the same delivery dose. In some embodiments, the administration by topical route increases the dermal bioavailability of the tofacitinib by about 20-fold compared to the dermal bioavailability of the tofacitinib of a comparative composition administered by oral route at the same delivery dose. In some embodiments, the administration by topical route increases the dermal bioavailability of the tofacitinib by about 30-fold compared to the dermal bioavailability of the tofacitinib of a comparative composition administered by oral route at the same delivery dose. In some embodiments, the administration by topical route increases the dermal bioavailability of the tofacitinib by about 40-fold compared to the dermal bioavailability of the tofacitinib of a comparative composition administered by oral route at the same delivery dose. In some embodiments, the administration by topical route increases the dermal bioavailability of the tofacitinib by about 50-fold compared to the dermal bioavailability of the tofacitinib of a comparative composition administered by oral route at the same delivery dose. In some embodiments, the administration by topical route increases the dermal bioavailability of the tofacitinib by about 60-fold compared to the dermal bioavailability of the tofacitinib of a comparative composition administered by oral route at the same delivery dose. In some embodiments, the administration by topical route increases the dermal bioavailability of the tofacitinib by about 70-fold compared to the dermal bioavailability of the tofacitinib of a comparative composition administered by oral route at the same delivery dose. In some embodiments, the administration by topical route increases the dermal bioavailability of the tofacitinib by about 80-fold compared to the dermal bioavailability of the tofacitinib of a comparative composition administered by oral route at the same delivery dose. In some embodiments, the administration by topical route increases the dermal bioavailability of the tofacitinib by about 100-fold compared to the dermal bioavailability of the tofacitinib of a comparative composition administered by oral route at the same delivery dose. In some embodiments, the administration by topical route increases the dermal bioavailability of the tofacitinib by about 200-fold compared to the dermal bioavailability of the tofacitinib of a comparative composition administered by oral route at the same delivery dose. In some embodiments, the administration by topical route increases the dermal bioavailability of the tofacitinib by about 300-fold compared to the dermal bioavailability of the tofacitinib of a comparative composition administered by oral route at the same delivery dose. In some embodiments, the administration by topical route increases the dermal bioavailability of the tofacitinib by about 400-fold compared to the dermal bioavailability of the tofacitinib of a comparative composition administered by oral route at the same delivery dose. In some embodiments, the administration by topical route increases the dermal bioavailability of the tofacitinib by about 500-fold compared to the dermal bioavailability of the tofacitinib of a comparative composition administered by oral route at the same delivery dose. In some embodiments, the administration by topical route increases the dermal bioavailability of the tofacitinib by about 600-fold compared to the dermal bioavailability of the tofacitinib of a comparative composition administered by oral route at the same delivery dose. In some embodiments, the administration by topical route increases the dermal bioavailability of the tofacitinib by about 700-fold compared to the dermal bioavailability of the tofacitinib of a comparative composition administered by oral route at the same delivery dose. In some embodiments, the administration by topical route increases the dermal bioavailability of the tofacitinib by about 800-fold compared to the dermal bioavailability of the tofacitinib of a comparative composition administered by oral route at the same delivery dose. In some embodiments, the administration by topical route increases the dermal bioavailability of the tofacitinib by about 900-fold compared to the dermal bioavailability of the tofacitinib of a comparative composition administered by oral route at the same delivery dose. In some embodiments, the administration by topical route increases the dermal bioavailability of the tofacitinib by about 1000-fold compared to the dermal bioavailability of the tofacitinib of a comparative composition administered by oral route at the same delivery dose. In some embodiments, the administration by topical route increases the dermal bioavailability of the tofacitinib by about 100-fold compared to the dermal bioavailability of the tofacitinib of a comparative composition administered by oral route at the same delivery dose.

### Improved Pharmacokinetic Parameters Upon Administration of Tofacitinib by Topical Route

Dermal bioavailability includes the following exemplary pharmacokinetic factors: rate (or time after administration) of achievement of minimum effective drug serum concentration (MEC), maximum drug serum concentration (Cmax), rate (or time after administration) of achievement of maximum drug serum concentration (Tmax), and the area under the drug serum concentration-time curve above a line representing minimum effective drug serum concentration (AUC). In some embodiments, the methods of treating a skin disease or condition by administration of tofacitinib by topical route, as described herein, leads to enhancement in one more of the factors mentioned above.

### I. Increased Cmax

In some embodiments, following administration of the composition by topical route the Cmax of the tofacitinib in skin increases by about 1-fold to about 1000-fold compared to the Cmax following administration of a comparative composition by oral route at the same delivery dose. In some embodiments, following administration of the composition by topical route the Cmax of the tofacitinib in skin increases by about 1-fold to about 500-fold compared to the Cmax following administration of a comparative composition by oral route at the same delivery dose. In some embodiments, following administration of the composition by topical route the Cmax of the tofacitinib in skin increases by about 1-fold to about 100-fold compared to the Cmax following administration of a comparative composition by oral route at the same delivery dose. In some embodiments, following administration of the composition by topical route the Cmax of the tofacitinib in skin increases by about 1-fold to about 50-fold compared to the Cmax following administration of a comparative composition by oral route at the same delivery dose. In some embodiments, following administration of the composition by topical route the Cmax of the tofacitinib in skin increases by about 1-fold to about 20-fold compared to the Cmax following administration of a comparative composition by oral route at the same delivery dose. In some embodiments, following administration of the composition by topical route the Cmax of the tofacitinib in skin increases by about 1-fold to about 10-fold compared to the Cmax following administration of a comparative composition by oral route at the same delivery dose.

In some embodiments, following administration of the composition by topical route the Cmax of the tofacitinib in skin increases by about 1-fold compared to the Cmax following administration of a comparative composition by oral route at the same delivery dose. In some embodiments, following administration of the composition by topical route the Cmax of the tofacitinib in skin increases by about 2-fold compared to the Cmax following administration of a comparative composition by oral route at the same delivery dose. In some embodiments, following administration of the composition by topical route the Cmax of the tofacitinib in skin increases by about 3-fold compared to the Cmax following administration of a comparative composition by oral route at the same delivery dose. In some embodiments, following administration of the composition by topical route the Cmax of the tofacitinib in skin increases by about 4-fold compared to the Cmax following administration of a comparative composition by oral route at the same delivery dose. In some embodiments, following administration of the composition by topical route the Cmax of the tofacitinib in skin increases by about 5-fold compared to the Cmax following administration of a comparative composition by oral route at the same delivery dose. In some embodiments, following administration of the composition by topical route the Cmax of the tofacitinib in skin increases by about 6-fold compared to the Cmax following administration of a comparative composition by oral route at the same delivery dose. In some embodiments, following administration of the composition by topical route the Cmax of the tofacitinib in skin increases by about 7-fold compared to the Cmax following administration of a comparative composition by oral route at the same delivery dose. In some embodiments, following administration of the composition by topical route the Cmax of the tofacitinib in skin increases by about 8-fold compared to the Cmax following administration of a comparative composition by oral route at the same delivery dose. In some embodiments, following administration of the composition by topical route the Cmax of the tofacitinib in skin increases by about 9-fold compared to the Cmax following administration of a comparative composition by oral route at the same delivery dose. In some embodiments, following administration of the composition by topical route the Cmax of the tofacitinib in skin increases by about 10-fold compared to the Cmax following administration of a comparative composition by oral route at the same delivery dose. In some embodiments, following administration of the composition by topical route the Cmax of the tofacitinib in skin increases by about 20-fold compared to the Cmax following administration of a comparative composition by oral route at the same delivery dose. In some embodiments, following administration of the composition by topical route the Cmax of the tofacitinib in skin increases by about 30-fold compared to the Cmax following administration of a comparative composition by oral route at the same delivery dose. In some embodiments, following administration of the composition by topical route the Cmax of the tofacitinib in skin increases by about 40-fold compared to the Cmax following administration of a comparative composition by oral route at the same delivery dose. In some embodiments, following administration of the composition by topical route the Cmax of the tofacitinib in skin increases by about 50-fold compared to the Cmax following administration of a comparative composition by oral route at the same delivery dose. In some embodiments, following administration of the composition by topical route the Cmax of the tofacitinib in skin increases by about 60-fold compared to the Cmax following administration of a comparative composition by oral route at the same delivery dose. In some embodiments, following administration of the composition by topical route the Cmax of the tofacitinib in skin increases by about 70-fold compared to the Cmax following administration of a comparative composition by oral route at the same delivery dose. In some embodiments, following administration of the composition by topical route the Cmax of the tofacitinib in skin increases by about 80-fold compared to the Cmax following administration of a comparative composition by oral route at the same delivery dose. In some embodiments, following administration of the composition by topical route the Cmax of the tofacitinib in skin increases by about 90-fold compared to the Cmax following administration of a comparative composition by oral route at the same delivery dose. In some embodiments, following administration of the composition by topical route the Cmax of the tofacitinib in skin increases by about 100-fold compared to the Cmax following administration of a comparative composition by oral route at the same delivery dose. In some embodiments, following administration of the composition by topical route the Cmax of the tofacitinib in skin increases by about 200-fold compared to the Cmax following administration of a comparative composition by oral route at the same delivery dose. In some embodiments, following administration of the composition by topical route the Cmax of the tofacitinib in skin increases by about 300-fold compared to the Cmax following administration of a comparative composition by oral route at the same delivery dose. In some embodiments, following administration of the composition by topical route the Cmax of the tofacitinib in skin increases by about 400-fold compared to the Cmax following administration of a comparative composition by oral route at the same delivery dose. In some embodiments, following administration of the composition by topical route the Cmax of the tofacitinib in skin increases by about 500-fold compared to the Cmax following administration of a comparative composition by oral route at the same delivery dose. In some embodiments, following administration of the composition by topical route the Cmax of the tofacitinib in skin increases by about 600-fold compared to the Cmax following administration of a comparative composition by oral route at the same delivery dose. In some embodiments, following administration of the composition by topical route the Cmax of the tofacitinib in skin increases by about 700-fold compared to the Cmax following administration of a comparative composition by oral route at the same delivery dose. In some embodiments, following administration of the composition by topical route the Cmax of the tofacitinib in skin increases by about 800-fold compared to the Cmax following administration of a comparative composition by oral route at the same delivery dose. In some embodiments, following administration of the composition by topical route the Cmax of the tofacitinib in skin increases by about 800-fold compared to the Cmax following administration of a comparative composition by oral route at the same delivery dose. In some embodiments, following administration of the composition by topical route the Cmax of the tofacitinib in skin increases by about 900-fold compared to the Cmax following administration of a comparative composition by oral route at the same delivery dose. In some embodiments, following administration of the composition by topical route the Cmax of the tofacitinib in skin increases by about 1000-fold compared to the Cmax following administration of a comparative composition by oral route at the same delivery dose.

### II. Increased AUC

In some embodiments, following administration of the composition by topical route the AUC of the tofacitinib in skin increases by about 1-fold to about 1000-fold compared to the AUC following administration of a comparative composition by oral route at the same delivery dose. In some embodiments, following administration of the composition by topical route the AUC of the tofacitinib in skin increases by about 1-fold to about 500-fold compared to the AUC following administration of a comparative composition by oral route at the same delivery dose. In some embodiments, following administration of the composition by topical route the AUC of the tofacitinib in skin increases by about 1-fold to about 100-fold compared to the AUC following administration of a comparative composition by oral route at the same delivery dose. In some embodiments, following administration of the composition by topical route the AUC of the tofacitinib in skin increases by about 1-fold to about 50-fold compared to the AUC following administration of a comparative composition by oral route at the same delivery dose. In some embodiments, following administration of the composition by topical route the AUC of the tofacitinib in skin increases by about 1-fold to about 20-fold compared to the AUC following administration of a comparative composition by oral route at the same delivery dose. In some embodiments, following administration of the composition by topical route the AUC of the tofacitinib in skin increases by about 1-fold to about 10-fold compared to the AUC following administration of a comparative composition by oral route at the same delivery dose.

In some embodiments, following administration of the composition by topical route the AUC of the tofacitinib in skin increases by about 1-fold compared to the AUC following administration of a comparative composition by oral route at the same delivery dose. In some embodiments, following administration of the composition by topical route the AUC of the tofacitinib in skin increases by about 2-fold compared to the AUC following administration of a comparative composition by oral route at the same delivery dose. In some embodiments, following administration of the composition by topical route the AUC of the tofacitinib in skin increases by about 3-fold compared to the AUC following administration of a comparative composition by oral route at the same delivery dose. In some embodiments, following administration of the composition by topical route the AUC of the tofacitinib in skin increases by about 4-fold compared to the AUC following administration of a comparative composition by oral route at the same delivery dose. In some embodiments, following administration of the composition by topical route the AUC of the tofacitinib in skin increases by about 5-fold compared to the AUC following administration of a comparative composition by oral route at the same delivery dose. In some embodiments, following administration of the composition by topical route the AUC of the tofacitinib in skin increases by about 6-fold compared to the AUC following administration of a comparative composition by oral route at the same delivery dose. In some embodiments, following administration of the composition by topical route the AUC of the tofacitinib in skin increases by about 7-fold compared to the AUC following administration of a comparative composition by oral route at the same delivery dose. In some embodiments, following administration of the composition by topical route the AUC of the tofacitinib in skin increases by about 8-fold compared to the AUC following administration of a comparative composition by oral route at the same delivery dose. In some embodiments, following administration of the composition by topical route the AUC of the tofacitinib in skin increases by about 9-fold compared to the AUC following administration of a comparative composition by oral route at the same delivery dose. In some embodiments, following administration of the composition by topical route the AUC of the tofacitinib in skin increases by about 10-fold compared to the AUC following administration of a comparative composition by oral route at the same delivery dose. In some embodiments, following administration of the composition by topical route the AUC of the tofacitinib in skin increases by about 20-fold compared to the AUC following administration of a comparative composition by oral route at the same delivery dose. In some embodiments, following administration of the composition by topical route the AUC of the tofacitinib in skin increases by about 30-fold compared to the AUC following administration of a comparative composition by oral route at the same delivery dose. In some embodiments, following administration of the composition by topical route the AUC of the tofacitinib in skin increases by about 40-fold compared to the AUC following administration of a comparative composition by oral route at the same delivery dose. In some embodiments, following administration of the composition by topical route the AUC of the tofacitinib in skin increases by about 50-fold compared to the AUC following administration of a comparative composition by oral route at the same delivery dose. In some embodiments, following administration of the composition by topical route the AUC of the tofacitinib in skin increases by about 60-fold compared to the AUC following administration of a comparative composition by oral route at the same delivery dose. In some embodiments, following administration of the composition by topical route the AUC of the tofacitinib in skin increases by about 70-fold compared to the AUC following administration of a comparative composition by oral route at the same delivery dose. In some embodiments, following administration of the composition by topical route the AUC of the tofacitinib in skin increases by about 80-fold compared to the AUC following administration of a comparative composition by oral route at the same delivery dose. In some embodiments, following administration of the composition by topical route the AUC of the tofacitinib in skin increases by about 90-fold compared to the AUC following administration of a comparative composition by oral route at the same delivery dose. In some embodiments, following administration of the composition by topical route the AUC of the tofacitinib in skin increases by about 100-fold compared to the AUC following administration of a comparative composition by oral route at the same delivery dose. In some embodiments, following administration of the composition by topical route the AUC of the tofacitinib in skin increases by about 200-fold compared to the AUC following administration of a comparative composition by oral route at the same delivery dose. In some embodiments, following administration of the composition by topical route the AUC of the tofacitinib in skin increases by about 300-fold compared to the AUC following administration of a comparative composition by oral route at the same delivery dose. In some embodiments, following administration of the composition by topical route the AUC of the tofacitinib in skin increases by about 400-fold compared to the AUC following administration of a comparative composition by oral route at the same delivery dose. In some embodiments, following administration of the composition by topical route the AUC of the tofacitinib in skin increases by about 500-fold compared to the AUC following administration of a comparative composition by oral route at the same delivery dose. In some embodiments, following administration of the composition by topical route the AUC of the tofacitinib in skin increases by about 600-fold compared to the AUC following administration of a comparative composition by oral route at the same delivery dose. In some embodiments, following administration of the composition by topical route the AUC of the tofacitinib in skin increases by about 700-fold compared to the AUC following administration of a comparative composition by oral route at the same delivery dose. In some embodiments, following administration of the composition by topical route the AUC of the tofacitinib in skin increases by about 800-fold compared to the AUC following administration of a comparative composition by oral route at the same delivery dose. In some embodiments, following administration of the composition by topical route the AUC of the tofacitinib in skin increases by about 800-fold compared to the AUC following administration of a comparative composition by oral route at the same delivery dose. In some embodiments, following administration of the composition by topical route the AUC of the tofacitinib in skin increases by about 900-fold compared to the AUC following administration of a comparative composition by oral route at the same delivery dose. In some embodiments, following administration of the composition by topical route the AUC of the tofacitinib in skin increases by about 1000-fold compared to the AUC following administration of a comparative composition by oral route at the same delivery dose.

### III. Decreased Tmax

In some embodiments, following administration of the composition by topical route the Tmax of the tofacitinib in skin decreases by about 1-fold to about 1000-fold compared to the Tmax following administration of a comparative composition by oral route at the same delivery dose. In some embodiments, following administration of the composition by topical route the Tmax of the tofacitinib in skin decreases by about 1-fold to about 500-fold compared to the Tmax following administration of a comparative composition by oral route at the same delivery dose. In some embodiments, following administration of the composition by topical route the Tmax of the tofacitinib in skin decreases by about 1-fold to about 100-fold compared to the Tmax following administration of a comparative composition by oral route at the same delivery dose. In some embodiments, following administration of the composition by topical route the Tmax of the tofacitinib in skin decreases by about 1-fold to about 50-fold compared to the Tmax following administration of a comparative composition by oral route at the same delivery dose. In some embodiments, following administration of the composition by topical route the Tmax of the tofacitinib in skin decreases by about 1-fold to about 20-fold compared to the Tmax following administration of a comparative composition by oral route at the same delivery dose. In some embodiments, following administration of the composition by topical route the Tmax of the tofacitinib in skin decreases by about 1-fold to about 10-fold compared to the Tmax following administration of a comparative composition by oral route at the same delivery dose.

In some embodiments, following administration of the composition by topical route the Tmax of the tofacitinib in skin decreases by about 1-fold compared to the Tmax following administration of a comparative composition by oral route at the same delivery dose. In some embodiments, following administration of the composition by topical route the Tmax of the tofacitinib in skin decreases by about 2-fold compared to the Tmax following administration of a comparative composition by oral route at the same delivery dose. In some embodiments, following administration of the composition by topical route the Tmax of the tofacitinib in skin decreases by about 3-fold compared to the Tmax following administration of a comparative composition by oral route at the same delivery dose. In some embodiments, following administration of the composition by topical route the Tmax of the tofacitinib in skin decreases by about 4-fold compared to the Tmax following administration of a comparative composition by oral route at the same delivery dose. In some embodiments, following administration of the composition by topical route the Tmax of the tofacitinib in skin decreases by about 5-fold compared to the Tmax following administration of a comparative composition by oral route at the same delivery dose. In some embodiments, following administration of the composition by topical route the Tmax of the tofacitinib in skin decreases by about 6-fold compared to the Tmax following administration of a comparative composition by oral route at the same delivery dose. In some embodiments, following administration of the composition by topical route the Tmax of the tofacitinib in skin decreases by about 7-fold compared to the Tmax following administration of a comparative composition by oral route at the same delivery dose. In some embodiments, following administration of the composition by topical route the Tmax of the tofacitinib in skin decreases by about 8-fold compared to the Tmax following administration of a comparative composition by oral route at the same delivery dose. In some embodiments, following administration of the composition by topical route the Tmax of the tofacitinib in skin decreases by about 9-fold compared to the Tmax following administration of a comparative composition by oral route at the same delivery dose. In some embodiments, following administration of the composition by topical route the Tmax of the tofacitinib in skin decreases by about 10-fold compared to the Tmax following administration of a comparative composition by oral route at the same delivery dose. In some embodiments, following administration of the composition by topical route the Tmax of the tofacitinib in skin decreases by about 20-fold compared to the Tmax following administration of a comparative composition by oral route at the same delivery dose. In some embodiments, following administration of the composition by topical route the Tmax of the tofacitinib in skin decreases by about 30-fold compared to the Tmax following administration of a comparative composition by oral route at the same delivery dose. In some embodiments, following administration of the composition by topical route the Tmax of the tofacitinib in skin decreases by about 40-fold compared to the Tmax following administration of a comparative composition by oral route at the same delivery dose. In some embodiments, following administration of the composition by topical route the Tmax of the tofacitinib in skin decreases by about 50-fold compared to the Tmax following administration of a comparative composition by oral route at the same delivery dose. In some embodiments, following administration of the composition by topical route the Tmax of the tofacitinib in skin decreases by about 60-fold compared to the Tmax following administration of a comparative composition by oral route at the same delivery dose. In some embodiments, following administration of the composition by topical route the Tmax of the tofacitinib in skin decreases by about 70-fold compared to the Tmax following administration of a comparative composition by oral route at the same delivery dose. In some embodiments, following administration of the composition by topical route the Tmax of the tofacitinib in skin decreases by about 80-fold compared to the Tmax following administration of a comparative composition by oral route at the same delivery dose. In some embodiments, following administration of the composition by topical route the Tmax of the tofacitinib in skin decreases by about 100-fold compared to the Tmax following administration of a comparative composition by oral route at the same delivery dose. In some embodiments, following administration of the composition by topical route the Tmax of the tofacitinib in skin decreases by about 200-fold compared to the Tmax following administration of a comparative composition by oral route at the same delivery dose. In some embodiments, following administration of the composition by topical route the Tmax of the tofacitinib in skin decreases by about 300-fold compared to the Tmax following administration of a comparative composition by oral route at the same delivery dose. In some embodiments, following administration of the composition by topical route the Tmax of the tofacitinib in skin decreases by about 400-fold compared to the Tmax following administration of a comparative composition by oral route at the same delivery dose. In some embodiments, following administration of the composition by topical route the Tmax of the tofacitinib in skin decreases by about 500-fold compared to the Tmax following administration of a comparative composition by oral route at the same delivery dose. In some embodiments, following administration of the composition by topical route the Tmax of the tofacitinib in skin decreases by about 600-fold compared to the Tmax following administration of a comparative composition by oral route at the same delivery dose. In some embodiments, following administration of the composition by topical route the Tmax of the tofacitinib in skin decreases by about 700-fold compared to the Tmax following administration of a comparative composition by oral route at the same delivery dose. In some embodiments, following administration of the composition by topical route the Tmax of the tofacitinib in skin decreases by about 800-fold compared to the Tmax following administration of a comparative composition by oral route at the same delivery dose. In some embodiments, following administration of the composition by topical route the Tmax of the tofacitinib in skin decreases by about 900-fold compared to the Tmax following administration of a comparative composition by oral route at the same delivery dose. In some embodiments, following administration of the composition by topical route the Tmax of the tofacitinib in skin decreases by about 1000-fold compared to the Tmax following administration of a comparative composition by oral route at the same delivery dose.

In some embodiments, the topical tofacitinib composition described herein provides improved stability or less degradation of the tofacitinib therein. In some embodiments, the [0088] In some embodiments, the topical tofacitinib composition described herein provides improved stability or less degradation of the tofacitinib therein. In some embodiments, the topical tofacitinib composition described herein are stable with respect to compound degradation (e.g. less than 30% degradation, less than 25% degradation, less than 20% degradation, less than 15% degradation, less than 10% degradation, less than 8% degradation, less than 5% degradation, less than 3% degradation, less than 2% degradation, or less than 1% degradation) over a period of any of at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 3 months, at least about 4 months, at least about 5 months, or at least about 6 months under storage conditions (e.g. room temperature). In other embodiments, the formulations described herein are stable with respect to tofacitinib degradation over a period of at least about 1 week. Also described herein are formulations that are stable with respect to tofacitinib degradation over a period of at least about 1 month.

### Dermal Pharmacokinetic Evaluation of Topical Formulations

Skin is the outermost largest organ in the body. It protects the body from physical, chemical, and microbial assaults and also prevents water loss from the body. The skin comprises three layers, i.e. epidermis, dermis, and subcutaneous tissue. Epidermis is made up of five layers composed of stratum corneum, stratum lucidum, stratum granulosum, stratum spinosum, and stratum basale. The stratum corneum is the outermost layer and comprise dead keratinocytes, and lamellar granules. The corneocytes and lipids form tight junctions in the stratum corneum like brick and mortar. Stratum corneum acts as a permeation and diffusional barrier for topically applied dosage form for skin disorders and transdermal drug delivery. The topical formulations are considered as a mainstay for the treatment of these skin disorders. Topical drug delivery systems are mostly preferred to deliver the drugs to the target site with minimal systemic adverse effects.

The bioavailability of the topical and transdermal administered drugs is affected by various factors. They include permeation through the skin (rate-limiting membrane stratum corneum), physicochemical properties of the drug (pKa, logP, solubility, molecular weight), excipients used in the formulation, type of formulation, presence of enzymes in skin structures, application site, and type of the skin. After the topical or transdermal application of the formulation onto the skin, the drug must permeate deeper layers by crossing various layers. Various permeability enhancement techniques are employed to increase drug transport into the stratum corneum. Consequently, skin cannot be deemed as a single compartment. The estimation of drug retained in skin layers is critical in topical and transdermal drug delivery. Therefore, there is a need to separate the skin into layers to estimate the drug permeation in different layers. Dermato-pharmacokinetic studies can be very useful for evaluation of topical product's performance and assessment in drug permeation-retention in the skin layers.

Many techniques may be used to assess dermato-pharmacokinetics. Parameters such as Cmax, Tmax, and AUC are extensively assessed in dermato-pharmacokinetic studies. Assessment of dermato-pharmacokinetics of administered topical formulations is desirable in appraising the products' safety and efficacy. Several techniques have been explored for the estimation of the drug in the different skin layers. In diseased skin, the stratum corneum barrier function is impaired, which does not hinder drug permeation. For dermato-pharmacokinetic studies, healthy skin may be more recommended than diseased skin. Using two or three techniques simultaneously provides superior benefits. Dermato-pharmacokinetic evaluation can be utilized for quality control tool for complex formulations and can provide the road map for newer product development.

### Dermato-Pharmacokinetic Evaluation Techniques

The dermato-pharmacokinetic method is employed to estimate drug concentrations in the skin layers. Although the stratum corneum might not be the site of action, it is the rate-limiting membrane for drug permeation. The dermato-pharmacokinetics is akin to blood and urine pharmacokinetic studies. The skin kinetics depends on the drug absorption and the termination of administration. Dermato-pharmacokinetics methods assume three mechanisms: i) stratum corneum behavior as the rate-limiting membrane in drug absorption; ii) diffusion of the drug into the dermis is directly proportional to the concentration of drug at the stratum corneum; iii) The drug concentrations in the skin layers (stratum corneum, epidermis, dermis) depict the dermatological efficacy of the drug [Citation2]. The parameters in dermato-pharmacokinetics include Cmax (maximum drug concentration in the skin layers), Tmax (time required to reach Cmax), and Area under the curve (AUC) in stratum corneum, epidermis, dermis. The Cmax, Tmax and AUC parameters are used to evaluate drug molecules' pharmacokinetics in the skin layers [Citation1]. The dermato-pharmacokinetic profile of the topically applied drugs is estimated using in-vitro and in-vivo techniques as discussed in the following section.

### Tape Stripping

Tape stripping is the most widely employed technique for the estimation of the drug in the stratum corneum. The utilization of tape stripping for quantification of drugs in the stratum corneum was proposed in the draft guidance by the United States Department of Health and Human Services in 1998 to assess the bioequivalence of the topical products. This method can be utilized in both in-vitro and in-vivo drug permeation studies for topical delivery systems applied to the skin. It is uncomplicated, easily implemented for quantification of drug retained in the stratum corneum. This technique is minimally invasive in the in-vivo conditions (Preclinical and clinical studies). It involves removing the stratum corneum layer one after another using adhesive tape strips. The tape strip is applied with the pressure using a roller to ensure uniform and proper adhesion onto the skin surface. The tape strip's application pressure should maintain uniform throughout the tape stripping process to get reproducible results. The collected tape strips are added to the solvent of interest in which the drug is highly soluble to extract the drug from the adhesive. The drug extracted is subjected to centrifugation and quantified using a validated analytical technique. The typical limitations with the tape stripping technique is the interference of adhesive, and the number of strips required to remove the stratum corneum is varied from person to person and site of application. There is a requirement of validating the procedure before the utilization of the process in preclinical and clinical studies. The number of stratum corneum layers differs in animals (mice, rat, rabbit, guinea pig, goat, pig) and humans.

The tape stripping technique is validated using the weighing method or the protein estimation method. In weighing method, the tape strip's weight is noted before and after tape stripping, and the protein levels of corneocytes are measured to determine the stratum corneum layers in the skin. Olesen et al. reported that protein removal was decreased with an increase in tape stripping depth. The study results revealed that tape stripping can be used to skin barrier research without discomfort and scars compared to skin biopsies.

### Microdialysis and Open-Flow Microperfusion

A microdialysis and open-flow microperfusion techniques are employed to assess the bioequivalence of the topical drug product. The drug concentration is determined in the dermis and hypodermis by implanting a semipermeable membrane probe guided by a needle. The probe is perfused with the sterile buffer, which mimics the blood vessels, equilibrates by interstitial fluids, and the drug molecules diffuse from tissues to the buffer. Isotonic saline/Ringer's lactate solution is perfused at the rate of 1-5 μL. FIG. 2 depicts the microdialysis and tape striping technique.

Figure below illustrates microdialysis and tape striping technique: (a) In microdialysis technique, the dialysis tubing is inserted into tissues and physiological solution (saline/ringer solution) is perfused at slow speed. The permeated drug molecules diffuse into the dialysis tube are collected for estimation of drug; (B) in tape stripping technique, the adhesive tape is used to strip the stratum corneum. The adhesive tape strip with stratum corneum is added to the solvent with high drug solubility. The drug is extracted, and the drug is estimated

### Vasoconstrictor Assay

Vasoconstrictor assay is also termed as the skin blanching assay, which is only limited to the corticosteroid formulations' bioavailability assessment. After applying corticosteroids, the local vasoconstriction can be measured by visual examination, chromametry, and analysis using digital imaging. In chromametry, the white light gets reflected into three different constraints red-green, yellow-blue, and light-dark. Using a digital image of 0.5 cm² with 300 dots per inch is analyzed for the above constraints. Visual inspection is an alternative to chromametry; the visual responses are given in the range of 0-4. Whereas 0 indicates no blanching, and required further no blanching, whereas numbers from 1 to 4 show the increasing array of blanching (absent=0, faint=1, faint-moderate=2, moderate-strong=3, and strong-intense blanching=4) practical application.

### Microscopic and Spectroscopic Methods

Along with the above methods, microscopic and spectroscopic techniques have been widely explored for the assessment of drug distribution. In microscopy analysis techniques, the dye (fluorescein, Nile red, and 5-bromodeoxyuridine, coumarin c6) is utilized for the assessment of the distribution and penetration into deeper layers of the skin. The dye may not exhibit similar behavior as a drug, which is the major limitation of the technique. In spectroscopic techniques, Attenuated Total Reflectance (ATR) or Fourier-Transform Infrared Spectroscopy (FTIR) is employed to estimate the modifications in the stratum corneum modifications. The conformation of proteins in outer skin layers, an assortment of lipids, treatment with chemical enhancers, and employing physical methods may modify permeability. The shift in the infrared bands illustrates the relative alterations between treated and untreated skin. The combination of Raman spectroscopy and confocal microscopy attracted great attention in estimating drug distribution in skin samples. The interference of water in Raman spectroscopy is null; it works on the principle of inelastic scattering of monochromatic light from a laser beam. The chemical structure, electronic configuration, and molecular bonds of detailed compound information are obtained using Raman spectroscopy.

### NON-LIMITING EMBODIMENTS

The following specific, non-limiting embodiments are to be construed as merely illustrative, and do not limit the present disclosure of the scope of the disclosure. Without further elaboration, it is believed that one skilled in the art can, based on the description herein, utilize the present disclosure to its fullest extent.

1. A topical composition, comprising from about 1 wt % to about 10 wt % of tofacitinib or a pharmaceutically acceptable salt thereof; from about 1 wt % to about 30 wt % of an ionic liquid comprising choline cations and geranic acid anions; and a topically acceptable carrier, wherein topical composition allows a therapeutically effective amount of tofacitinib to reach a target depth within or beyond the skin.

2. The composition of Embodiment 1, wherein the pharmaceutical composition comprises from about 1 wt % to about 10 wt % of tofacitinib or a pharmaceutically acceptable salt thereof.

3. The composition of Embodiment 1, wherein the pharmaceutical composition comprises from about 1 wt % to about 8 wt % of tofacitinib or a pharmaceutically acceptable salt thereof.

4. The composition of Embodiment 1, wherein the pharmaceutical composition comprises from about 1 wt % to about 5 wt % of tofacitinib or a pharmaceutically acceptable salt thereof.

5. The composition of Embodiment 1, wherein the pharmaceutical composition comprises about 1 wt % of tofacitinib or a pharmaceutically acceptable salt thereof.

6. The composition of Embodiment 1, wherein the pharmaceutical composition comprises about 2 wt % of tofacitinib or a pharmaceutically acceptable salt thereof.

7. The composition of Embodiment 1, wherein the pharmaceutical composition comprises about 3 wt % of tofacitinib or a pharmaceutically acceptable salt thereof.

8. The composition of Embodiment 1, wherein the pharmaceutical composition comprises about 4 wt % of tofacitinib or a pharmaceutically acceptable salt thereof.

9. The composition of Embodiment 1, wherein the pharmaceutical composition comprises about 5 wt % of tofacitinib or a pharmaceutically acceptable salt thereof.

10. The composition of Embodiment 1-9, wherein the pharmaceutical composition comprises from about 2.5 wt % to about 30 wt % of the ionic liquid.

11. The composition of Embodiment 1-9, wherein the pharmaceutical composition comprises from about wt % to about 30 wt % of the ionic liquid.

12. The composition of Embodiment 1-9, wherein the pharmaceutical composition comprises from about 7.5 wt % to about 30 wt % of the ionic liquid.

13. The composition of Embodiment 1-9, wherein the pharmaceutical composition comprises from about wt % to about 30 wt % of the ionic liquid.

14. The composition of Embodiment 1-9, wherein the pharmaceutical composition comprises from about 12.5 wt % to about 30 wt % of the ionic liquid.

15. The composition of Embodiment 1-9, wherein the pharmaceutical composition comprises from about wt % to about 30 wt % of the ionic liquid.

16. The composition of Embodiment 1-9, wherein the pharmaceutical composition comprises from about 17.5 wt % to about 30 wt % of the ionic liquid.

17. The composition of Embodiment 1-9, wherein the pharmaceutical composition comprises from about wt % to about 30 wt % of the ionic liquid.

18. The composition of Embodiment 1-9, wherein the pharmaceutical composition comprises from about 20 wt % to about 25 wt % of the ionic liquid.

19. The composition of Embodiment 1-9, wherein the pharmaceutical composition comprises from about wt % to about 30 wt % of the ionic liquid.

20. The composition of Embodiment 1-9, wherein the pharmaceutical composition comprises about 25 wt % of the ionic liquid.

21. The composition of Embodiment 1-20, wherein the composition is a topical ointment.

22. The composition of Embodiment 21, wherein the composition comprises propylene glycol.

23. The composition of Embodiment 22, wherein the composition comprises from about 5 wt % to about 20 wt % of propylene glycol.

24. The composition of Embodiment 21-23, wherein the composition comprises glycerin.

25. The composition of Embodiment 22, wherein the composition comprises from about 20 wt % to about 30 wt % of glycerin.

26. The composition of Embodiment 21-25, wherein the composition comprises polyethylene glycol.

27. The composition of Embodiment 26, wherein the composition comprises from about 30 wt % to about 40 wt % of polyethylene glycol.

28. The composition of Embodiment 26-27, wherein the polyethylene glycol is Polyethylene Glycol 3350.

29. The composition of Embodiment 21-28, wherein the composition comprises diethylene glycol monoethyl ether.

30. The composition of Embodiment 29, wherein the composition comprises from about 1 wt % to about 15 wt % of diethylene glycol monoethyl ether.

31. The composition of Embodiment 21-30, wherein the composition comprises butylated hydroxyanisole.

32. The composition of Embodiment 31, wherein the composition comprises from about 0.01 wt % to about 0.1 wt % of butylated hydroxyanisole.

33. The composition of Embodiment 21-32, wherein the composition comprises titanium dioxide.

34. The composition of Embodiment 33, wherein the composition comprises from about 0.1 wt % to about 1 wt % of titanium dioxide.

35. The composition of Embodiment 1-20, wherein the composition is a topical gel.

36. The composition of Embodiment 35, wherein the composition comprises diethylene glycol monoethyl ether.

37. The composition of Embodiment 36, wherein the composition comprises from about 10 wt % to about 45 wt % of diethylene glycol monoethyl ether.

38. The composition of Embodiment 35-37, wherein the composition comprises polyethylene glycol.

39. The composition of Embodiment 38, wherein the composition comprises from about 10 wt % to about 35 wt % of polyethylene glycol.

40. The composition of Embodiment 39, wherein the polyethylene glycol is Polyethylene Glycol 400.

41. The composition of Embodiment 35-40, wherein the composition comprises isopropyl myristate.

42. The composition of Embodiment 41, wherein the composition comprises from about 5 wt % to about 35 wt % of isopropyl myristate.

43. The composition of Embodiment 35-42, wherein the composition comprises peppermint oil.

44. The composition of Embodiment 43, wherein the composition comprises from about 0.01 wt % to about 0.20 wt % of peppermint oil.

45. The composition of Embodiment 35-44, wherein the composition comprises butylated hydroxyanisole.

46. The composition of Embodiment 45, wherein the composition comprises from about 0.01 wt % to about 0.1 wt % of butylated hydroxyanisole.

47. The composition of Embodiment 35-46, wherein the composition comprises Polyamide-3.

48. The composition of Embodiment 47, wherein the composition comprises from about 0.1 wt % to about 20 wt % of Polyamide-3.

49. The composition of Embodiment 35-48, wherein the composition comprises water.

50. The composition of Embodiment 49, wherein the composition comprises from about 0.1 wt % to about 5 wt % of water.

51. The composition of any one of Embodiment 1-4, wherein the target depth is from about 0.05 mm to about 20 mm.

52. The composition of any one of Embodiment 1-4, wherein the target depth is from about 0.1 mm to about 15 mm.

53. The composition of any one of Embodiment 1-4, wherein the target depth is from about 1 mm to about 10 mm.

54. The composition of any one of Embodiment 1-7, wherein the pharmaceutical composition allows therapeutically effective amount of tofacitinib to reach at least an epidermis layer.

55. The composition of any one of Embodiment 1-8, wherein the pharmaceutical composition allows therapeutically effective amount of tofacitinib to reach at least a dermis layer.

56. The composition of any one of Embodiment 1-9, wherein the pharmaceutical composition allows therapeutically effective amount of tofacitinib to reach at least a subcutaneous tissue layer.

57. The composition of any one of Embodiments 1-10, wherein the pharmaceutical composition allows therapeutically effective amount of tofacitinib to reach at least a muscle tissue.

58. The composition of any one of Embodiments 1-22, wherein the ionic liquid is a deep eutectic solvent

(DES).

59. The composition of any one of Embodiments 1-23, wherein the ionic liquid comprises the choline cation and fatty acid anion in a molar ratio in a range of 1:1 to 1:4 of choline cation to geranic acid anion.

60. The composition of any one of Embodiments 1-23, wherein the ionic liquid comprises the choline cation and fatty acid anion in a molar ratio of 1:2 of choline cation to geranic acid anion.

61. The topical composition of Embodiment 1-60, wherein the administration by topical route increases the dermal bioavailability of tofacitinib by about 1.5-fold to about 1000-fold compared to the dermal bioavailability of the substituted or unsubstituted diindolylmethane of a comparative composition administered by oral route at the same delivery dose.

62. The topical composition of Embodiment 1-60, wherein the administration by topical route increases the dermal bioavailability of tofacitinib by about 1.5-fold to about 500-fold compared to the dermal bioavailability of the substituted or unsubstituted diindolylmethane of a comparative composition administered by oral route at the same delivery dose.

63. The topical composition of Embodiment 1-60, wherein the administration by topical route increases the dermal bioavailability of tofacitinib by about 1.5-fold to about 200-fold compared to the dermal bioavailability of the substituted or unsubstituted diindolylmethane of a comparative composition administered by oral route at the same delivery dose.

64. The topical composition of Embodiment 1-60, wherein the administration by topical route increases the dermal bioavailability of tofacitinib by about 1.5-fold to about 100-fold compared to the dermal bioavailability of the substituted or unsubstituted diindolylmethane of a comparative composition administered by oral route at the same delivery dose.

65. The topical composition of Embodiment 1-60, wherein the administration by topical route increases the dermal bioavailability of tofacitinib by about 1.5-fold to about 50-fold compared to the dermal bioavailability of the substituted or unsubstituted diindolylmethane of a comparative composition administered by oral route at the same delivery dose.

66. The topical composition of Embodiment 1-60, wherein the administration by topical route increases the dermal bioavailability of tofacitinib by about 1.5-fold to about 20-fold compared to the dermal bioavailability of the substituted or unsubstituted diindolylmethane of a comparative composition administered by oral route at the same delivery dose.

67. The topical composition of Embodiment 1-60, wherein the administration by topical route increases the dermal bioavailability of tofacitinib by about 1.5-fold to about 15-fold compared to the dermal bioavailability of the substituted or unsubstituted diindolylmethane of a comparative composition administered by oral route at the same delivery dose.

68. The topical composition of Embodiment 1-60, wherein the administration by topical route increases the dermal bioavailability of tofacitinib by about 1.5-fold to about 10-fold compared to the dermal bioavailability of the substituted or unsubstituted diindolylmethane of a comparative composition administered by oral route at the same delivery dose.

69. The topical composition of Embodiment 1-60, wherein the administration by topical route increases the dermal bioavailability of tofacitinib by about 1.5-fold to about 5-fold compared to the dermal bioavailability of the substituted or unsubstituted diindolylmethane of a comparative composition administered by oral route at the same delivery dose.

70. The topical composition of Embodiment 1-60, wherein the administration by topical route increases the dermal bioavailability of tofacitinib by about 1.5-fold to about 3-fold compared to the dermal bioavailability of the substituted or unsubstituted diindolylmethane of a comparative composition administered by oral route at the same delivery dose.

71. The topical composition of Embodiment 1-70, wherein following administration of the composition by topical route the Cmax of tofacitinib in skin increases by about 1.5-fold to about 1000-fold compared to the Cmax following administration of a comparative composition by oral route at the same delivery dose.

72. The topical composition of Embodiment 1-70, wherein following administration of the composition by topical route the Cmax of tofacitinib in skin increases by about 1.5-fold to about 500-fold compared to the Cmax following administration of a comparative composition by oral route at the same delivery dose.

73. The topical composition of Embodiment 1-70, wherein following administration of the composition by topical route the Cmax of tofacitinib in skin increases by about 1.5-fold to about 200-fold compared to the Cmax following administration of a comparative composition by oral route at the same delivery dose.

74. The topical composition of Embodiment 1-70, wherein following administration of the composition by topical route the Cmax of tofacitinib in skin increases by about 1.5-fold to about 100-fold compared to the Cmax following administration of a comparative composition by oral route at the same delivery dose.

75. The topical composition of Embodiment 1-70, wherein following administration of the composition by topical route the Cmax of tofacitinib in skin increases by about 1.5-fold to about 50-fold compared to the Cmax following administration of a comparative composition by oral route at the same delivery dose.

76. The topical composition of Embodiment 1-70, wherein following administration of the composition by topical route the Cmax of tofacitinib in skin increases by about 1.5-fold to about 20-fold compared to the Cmax following administration of a comparative composition by oral route at the same delivery dose.

77. The topical composition of Embodiment 1-70, wherein following administration of the composition by topical route the Cmax of tofacitinib in skin increases by about 1.5-fold to about 15-fold compared to the Cmax following administration of a comparative composition by oral route at the same delivery dose.

78. The topical composition of Embodiment 1-70, wherein following administration of the composition by topical route the Cmax of tofacitinib in skin increases by about 1.5-fold to about 10-fold compared to the Cmax following administration of a comparative composition by oral route at the same delivery dose.

79. The topical composition of Embodiment 1-70, wherein following administration of the composition by topical route the Cmax of tofacitinib in skin increases by about 1.5-fold to about 5-fold compared to the Cmax following administration of a comparative composition by oral route at the same delivery dose.

80. The topical composition of Embodiment 1-70, wherein following administration of the composition by topical route the Cmax of tofacitinib in skin increases by about 1.5-fold to about 3-fold compared to the Cmax following administration of a comparative composition by oral route at the same delivery dose.

81. The topical composition of Embodiment 1-80, wherein following administration of the composition by topical route the AUC of tofacitinib in skin increases by about 1.5-fold to about 1000-fold compared to the AUC following administration of a comparative composition by oral route at the same delivery dose.

82. The topical composition of Embodiment 1-80, wherein following administration of the composition by topical route the AUC of tofacitinib in skin increases by about 1.5-fold to about 500-fold compared to the AUC following administration of a comparative composition by oral route at the same delivery dose.

83. The topical composition of Embodiment 1-80, wherein following administration of the composition by topical route the AUC of tofacitinib in skin increases by about 1.5-fold to about 200-fold compared to the AUC following administration of a comparative composition by oral route at the same delivery dose.

84. The topical composition of Embodiment 1-80, wherein following administration of the composition by topical route the AUC of tofacitinib in skin increases by about 1.5-fold to about 100-fold compared to the AUC following administration of a comparative composition by oral route at the same delivery dose.

85. The topical composition of Embodiment 1-80, wherein following administration of the composition by topical route the AUC of tofacitinib in skin increases by about 1.5-fold to about 50-fold compared to the AUC following administration of a comparative composition by oral route at the same delivery dose.

86. The topical composition of Embodiment 1-80, wherein following administration of the composition by topical route the AUC of tofacitinib in skin increases by about 1.5-fold to about 20-fold compared to the AUC following administration of a comparative composition by oral route at the same delivery dose.

87. The topical composition of Embodiment 1-80, wherein following administration of the composition by topical route the AUC of tofacitinib in skin increases by about 1.5-fold to about 15-fold compared to the AUC following administration of a comparative composition by oral route at the same delivery dose.

88. The topical composition of Embodiment 1-80, wherein following administration of the composition by topical route the AUC of tofacitinib in skin increases by about 1.5-fold to about 10-fold compared to the AUC following administration of a comparative composition by oral route at the same delivery dose.

89. The topical composition of Embodiment 1-80, wherein following administration of the composition by topical route the AUC of tofacitinib in skin increases by about 1.5-fold to about 5-fold compared to the AUC following administration of a comparative composition by oral route at the same delivery dose.

90. The topical composition of Embodiment 1-80, wherein following administration of the composition by topical route the AUC of tofacitinib in skin increases by about 1.5-fold to about 3-fold compared to the AUC following administration of a comparative composition by oral route at the same delivery dose.

91. The topical composition of Embodiment 1-90, wherein following administration of the composition by topical route the Tmax of tofacitinib in skin decreases by about 1-fold to about 1000-fold compared to the Tmax following administration of a comparative composition by oral route at the same delivery dose.

92. The topical composition of Embodiment 1-90, wherein following administration of the composition by topical route the Tmax of tofacitinib in skin decreases by about 1-fold to about 500-fold compared to the Tmax following administration of a comparative composition by oral route at the same delivery dose.

93. The topical composition of Embodiment 1-90, wherein following administration of the composition by topical route the Tmax of tofacitinib in skin decreases by about 1-fold to about 200-fold compared to the Tmax following administration of a comparative composition by oral route at the same delivery dose.

94. The topical composition of Embodiment 1-90, wherein following administration of the composition by topical route the Tmax of tofacitinib in skin decreases by about 1-fold to about 100-fold compared to the Tmax following administration of a comparative composition by oral route at the same delivery dose.

95. The topical composition of Embodiment 1-90, wherein following administration of the composition by topical route the Tmax of tofacitinib in skin decreases by about 1-fold to about 50-fold compared to the Tmax following administration of a comparative composition by oral route at the same delivery dose.

96. The topical composition of Embodiment 1-90, wherein following administration of the composition by topical route the Tmax of tofacitinib in skin decreases by about 1-fold to about 20-fold compared to the Tmax following administration of a comparative composition by oral route at the same delivery dose.

97. The topical composition of Embodiment 1-90, wherein following administration of the composition by topical route the Tmax of tofacitinib in skin decreases by about 1-fold to about 15-fold compared to the Tmax following administration of a comparative composition by oral route at the same delivery dose.

98. The topical composition of Embodiment 1-90, wherein following administration of the composition by topical route the Tmax of tofacitinib in skin decreases by about 1-fold to about 10-fold compared to the Tmax following administration of a comparative composition by oral route at the same delivery dose.

99. The topical composition of Embodiment 1-90, wherein following administration of the composition by topical route the Tmax of tofacitinib in skin decreases by about 1-fold to about 5-fold compared to the Tmax following administration of a comparative composition by oral route at the same delivery dose.

100. The topical composition of Embodiment 1-90, wherein following administration of the composition by topical route the Tmax of tofacitinib in skin decreases by about 1-fold to about 3-fold compared to the Tmax following administration of a comparative composition by oral route at the same delivery dose.

101. The topical composition of Embodiment 1-100, wherein composition comprises tofacitinib.

102. The topical composition of Embodiment 1-100, wherein the composition comprises tofacitinib citrate.

### Non-Limiting Examples

The following specific, non-limiting examples are to be construed as merely illustrative, and do not limit the present disclosure of the scope of the disclosure. Without further elaboration, it is believed that one skilled in the art can, based on the description herein, utilize the present disclosure to its fullest extent.

### Example 1: Preparation of an Ionic Liquid Containing Choline Cation and Geranic Acid Anion in a 1:1 Molar Ratio (Ionic Liquid B)

The purified GMP Penta Geranic acid (311.0 g, 1.848 mol) was placed in a 2 L round bottomed flask. The flask was placed in a water bath at 20° C. and stirred. Then choline bicarbonate (381.7 g, 1.848 mol) 80% solution in water (Sigma, C7519, 209 ml) was added slowly (drop-wise) with an addition funnel, total addition time was 120 min. The flask was stirred overnight (12 hrs) to maximize the escape of the resulting CO₂. The flask was placed in the rotavap and the remaining CO₂ was removed at room temperature (20° C.) and a small vacuum (30 mbar). After no more CO₂ evolution was observed in the form of foam, the bath was heated to 60° C. and vacuum increased to −100 kPa to remove the resulting water. After no more water evaporation was observed by condensation on the dry ice trap of the rotavap, the flask was further heated at 60° C. and −100 kPa for 36 additional hrs to dry the final product. 475 g of product (94.7% yield) was obtained. HPLC analysis shows 97.9% purity.

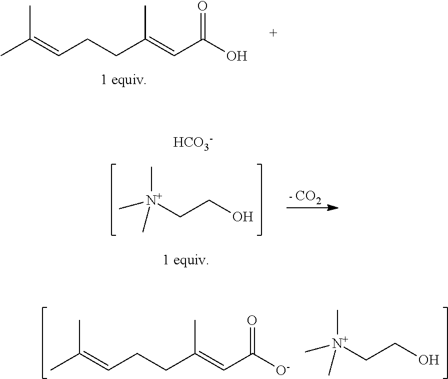

### Example 2: Preparation of an Ionic Liquid Containing Choline Cation and Geranic Acid Anion in a 1:2 Molar Ratio (Ionic Liquid A)

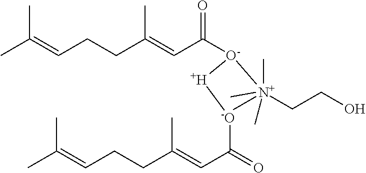

To two equivalents (9.88 g., 0.059 moles) of neat geranic acid, recrystallized 5× at −70° C. from 70% geranic acid/30% acetone, in a 500 mL round bottom flask was added one equivalent of choline bicarbonate (80 wt % solution, 6.06 g, 0.029 mol). The mixture was stirred at room temperature until no more CO₂ evolved. Solvent was removed by rotary evaporation at 60° C. for 20 min, and the product was dried in a vacuum oven for 48 h at 60° C.

Physical characterization at 25° C.: solubility in water=0.5 M; density=0.990 g/mL; conductivity=0.0431 mS/cm; viscosity=1345 cP.

### Example 3: Alternate Preparation of an Ionic Liquid Containing Choline Cation and Geranic Acid Anion in a 1:2 Molar Ratio (Ionic Liquid A)

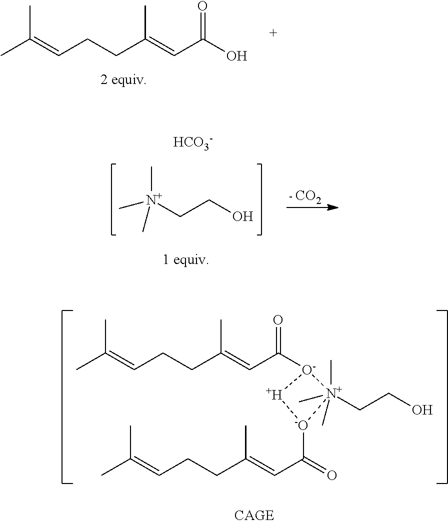

The purified GMP Penta Geranic acid (155 g, 0.921 mol) was placed in a 1 L round bottomed flask. The flask was placed in a water bath at 20° C. and stirred. Then choline bicarbonate (95.1 g, 0.460) 80% solution in water (Sigma, C7519, Lot #: 059K1526V, 209 ml) was added slowly (drop-wise) with an addition funnel, total addition time was 35 min. The flask was stirred overnight (12 hrs) to maximize the escape of the resulting CO₂. The flask was placed in the rotavap and the remaining CO₂ was removed at room temperature (20° C.) and a small vacuum (30 mbar). After no more CO₂ evolution was observed in the form of foam, the bath was heated to 60° C. and vacuum increased to −100 kPa to remove the resulting water. After no more water evaporation was observed by condensation on the dry ice trap of the rotavap, the flask was further heated at 60° C. and −100 kPa for 36 additional hrs to dry the final product. 197 g of Cage (96% yield) was obtained. 1H-NMR spectrum looks similar to the one of CB-0001. HPLC analysis shows 95.1% purity.

### Example 4: Preparation of a Topical Ointment Containing Tofacitinib and Ionic Liquid (Tofacitinib+IL Ointment)

Tofacitinib, Ionic Liquid A (according to Example 2), ointment base disclosed herein and other excipients are mixture together until a homogenous composition is achieved.

### Example 5: Preparation of a Topical Gel Containing Tofacitinib and Ionic Liquid (Tofacitinib+IL Gel)

Tofacitinib, Ionic Liquid A (according to Example 2), gelling agents, other excipients and water are mixture together until a homogenous composition is achieved.

### Example 6: Skin Flux Assay of Tofacitinib Compositions

Tofacitinib delivery by the following four compositions is measured using a static Franz cell setup (N=3 for each composition). Study was performed in Franz cells with human cadaver skin clamped over receptor chamber containing saline. The skin sample thickness was between 0.24-0.53 mm. The test composition was applied to surface of skin and occluded. After 24 h at 37° C., the surface of the skin sample was washed with saline and tape stripped to remove compositions remaining on the outer surface of the skin sample. The skin sample was then extracted in methanol: saline, 1:1, v/v. Dermis and epidermis layers were extracted together and not separated. Receptor is the saline underneath the skin that was sampled directly. The concentration of tofacitinib in these samples was quantified by HPLC. The skin flux evaluation of a topical tofacitinib gel composition according to certain embodiment is shown in FIG. 4

### Example 7: PK Comparison Study in Minipigs

Tofacitinib pharmacokinetic evaluation was conducted according to the following protocol.

### Tofacitinib in Minipigs Study Protocol

Female Göttingen Minipigs dosed with three tofacitinib formulations to compare PK and topical exposure to tofacitinib. N=3 each group.
   Low-Dose Topical (1% Tofacitinib CGB-500 Ointment): total drug dose=5 mg
   High-Dose Topical (3% Tofacitinib CGB-500 Ointment): total drug dose=15 mg
   Oral Reference Suspension (0.5% Tofacitinib Suspension): total drug dose=5 mg
   Minipigs dosed QD for 7 days. Plasma samples drawn on Day 1 and Day 7.
Skin biopsies taken on Day 7 from all groups
   Tape stripping (N=10) performed to remove stratum corneum (SC)
      2 punches from dosing area and 1 from non-dosing area
   Dermis and epidermis separated by scalpel

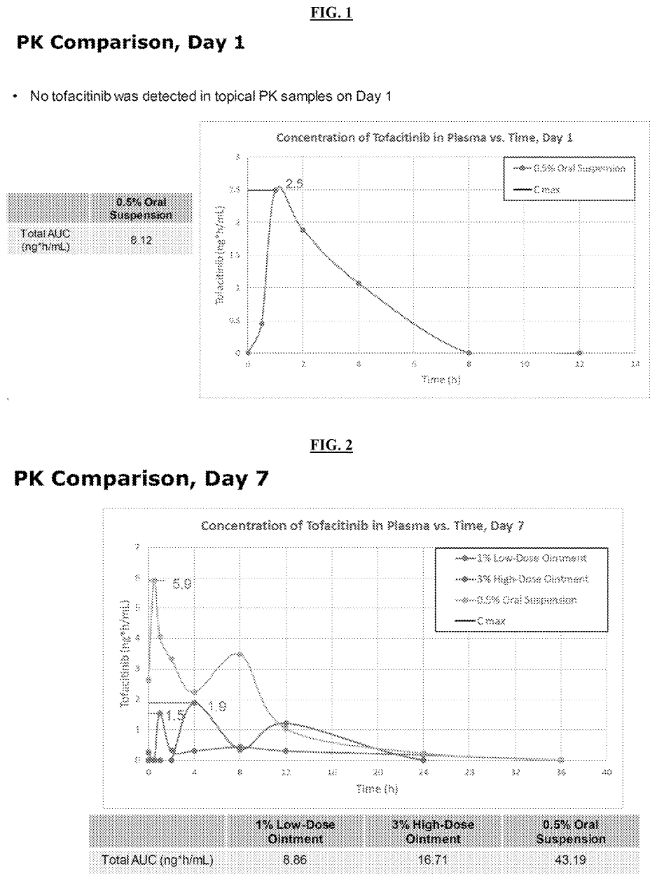

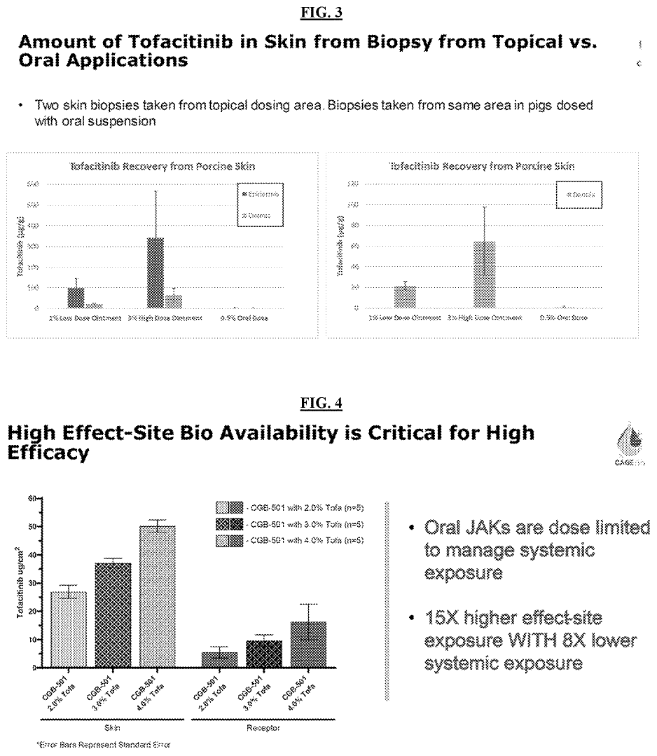

The results were summarized in FIGS. 1-3, showing improved drug delivery to the skin for the topical tofacitinib compositions as compared to the oral composition.

### Example 8: Improved Efficacy of Atopic Dermatitis Treatment by Topical Administration of Tofacitinib Ointment (CGB-500)

A Phase IIa clinical study was conducted based on the study protocol below.

Change from Baseline in Peak lesional Pruritus
NRS score at Week 4, Week 8, and Week 12.
Time to achieve Peak lesional Pruritus NRS score
improvement of at least 2, 3, or 4 points.

Methodology:
This is 2-center, randomized, double-blind, vehicle-controlled, parallel group study to evaluate the tolerability and
effectiveness of CGB-500 for the treatment of atopic dermatitis.
Subjects who give written informed consent will be screened for the study. Eligible subjects will be randomized
(Day 0) in a 1:1 ratio to twice daily topical application of CGB-500 or Vehicle Ointment for 8 weeks.
The anticipated duration of each subject's participation in the study is approximately 12 weeks. Screening and
Baseline will be on Day 0, followed by an 8-week double-blind treatment period: Visit 2 (Day 14/Week 2), Visit 3
(Day 28/Week 4), and Visit 4 (Day 56/Week 8, end of treatment [EOT]). A follow-up visit (Visit 5) will be
conducted at Day 84/Week 12 (end-of study [EOS]).
Number of Subjects:
Approximately 44 subjects will be screened so that 40 subjects will be enrolled and randomized.
Subject Population:
Inclusion criteria
1. Outpatient, male or female of any race, 18 years of age or older. Female subjects of childbearing potential must
have a negative urine pregnancy test at Baseline and practice a reliable method of contraception throughout the
study (Appendix 1).
2. Have a clinical diagnosis of atopic dermatitis for at least 6 months prior to Day 0 (at the Screening and Baseline
visit) that has been clinically stable for ≥3 months prior to dose administration, and is confirmed to be atopic
dermatitis according to the criteria of Hanifin and Rajka.
3. Have an IGA score of 2 (mild) or 3 (moderate) at Day 0.
4. Have an EASI score of 1.1 to 12.0 (i.e., mild or moderate).
5. Have atopic dermatitis on the arms and other body parts covering at least 2% of total body surface area (BSA)
and up to and including 12% of total BSA at Day 0.
6. Have a presence of 1 to 3 target atopic dermatitis lesions of total surface area of 15 to 30 cm², located on a body
part easily visible to the subject and the investigator. This is the target lesion(s) that will be treated in this study.
7. Agree to use only low potency corticosteroid (up to 1% hydrocortisone) topical cream or ointment for the other
affected parts of the body that are not being treated with the investigational product in the study.
8. In general, good health as determined by medical history and physical examination at the time of screening
(Investigator discretion).
9. Have a Peak Pruritus NRS score of ≥30 on the scale of 0 to 100, especially on the target lesions.
10. Ability to follow study instructions and likely to complete all required visits.
11. Have signed the Institutional Review Board (IRB)-approved informed consent form (ICF) prior to any study-
related procedures being performed.
12. Participating in any other clinical trial from 30 days prior to Screening and throughout the planned 12-week
study period.
Exclusion criteria
1. Female subjects who are pregnant, breast-feeding, or of childbearing potential and not practicing reliable birth
control.
2. Known hypersensitivity of previous allergic reaction to any constituent of the Investigational Product (i.e.,
essential oils, choline, phosphatidylcholine, glycerol, propylene glycol, polyethylene glycol).
3. Any transient flushing syndrome.
4. Severe atopic dermatitis.
5. Unstable course of atopic dermatitis (spontaneous improvement over time).
6. Skin infections (bacterial, fungal or viral) that can interfere with reliable atopic dermatitis assessments.
7. Basal cell carcinoma within 6 months of Baseline (Visit 1).
8. History of confounding skin conditions, e.g., psoriasis, rosacea, erythroderma, or ichthyosis or presence of
Netherton's syndrome, immunological deficiencies or diseases. HIV, diabetes, malignancy, serious active of
recurrent infection, clinically significant severe renal insufficiency, or severe hepatic disorders.
9. Use within 1 month prior to Baseline of 1) oral or intravenous corticosteroids, 2) UVA/UVB therapy, 3) PUVA
(psoralen plus ultraviolet A) therapy, 4) tanning booths, 5) non-prescription UV light sources,
6) immunomodulators or immunosuppressive therapies, 7) interferon, 8) cytotoxic drugs, 9) crisaborole,
10) pimecrolimus, or 11) injectable biologics (e.g. Dupixent)
10. Use within 14 days of Baseline of: 1) systemic antibiotics, 2) systemic JAK products, 3) calcipotriene or other
vitamin D preparations, or 4) retinoids.
11. Use within 7 days prior to Baseline of: 1) antihistamines, 2) topical antibiotics, 3) topical corticosteroids or
4) other topical drug products.
12. Use within 24 hours prior to Baseline of any topical product (e.g., sunscreens, lotions, creams, emollients,
moisturizers) in the areas to be treated.
13. Known allergy or hypersensitivity to tofacitinib or any other component of the Investigational Product (i.e.,
essential oils, choline, phosphatidylcholine, glycerol, propylene glycol, polyethylene glycol).
14. Uncontrolled systemic disease.
15. Foreseen unprotected and intense/excessive UV exposure during the study.
16. Scheduled or planned surgical procedures during the study.
17. Unable or unwilling to comply with any of the study requirements.
18. Medical or psychiatric conditions, or a personal situation, that may increase the risk associated with study
participation or may interfere with interpretation of study results or subject compliance and, in the opinion of
the Principal Investigator, makes the subject inappropriate for study entry.
19. Clinically significant alcohol or drug abuse, or history of poor cooperation or unreliability.
Test product, dose and form of administration:
Investigational Product (CGB-500) will be applied topically only to the same lesion(s) with a total surface area of
15 to 30 cm² twice daily, once in the morning and once in the evening, for a period of 8 weeks. The target lesion(s)
will be selected at Baseline and will be properly marked by the Investigator. The subject will be instructed that only
these specific areas are to be treated and evaluated in this study. If necessary, the Investigator may reduce the dose
by reducing the frequency of application.
Reference therapy, dose and mode of administration:
Vehicle ointment will be applied following the same procedures as for CGB-500.
Duration of treatment:
8 weeks.
Statistics:
Efficacy: All statistical processing will be described in the Statistical analysis plan (SAP). All statistical rests will
be 2-sided and will be performed at the 0.05 level of significance. Descriptive statistics will be used to provide an
overview of the efficacy and safety results. For categorical parameters, the number and percentage of subjects in
each category will be presented. For continuous parameters, descriptive statistics will include n (number of
subjects), mean, standard deviation, median, minimum, and maximum. Appropriate inferential statistics will be
used for the primary and secondary efficacy variables.
Safety: Adverse events will be tabulated according to the current version of the Medical Dictionary for Regulatory
Activities (MedDRA). Frequencies and percentages will be presented. All TEAEs will be summarized by the
number of subjects reporting each TEAE, by system organ class and preferred term.

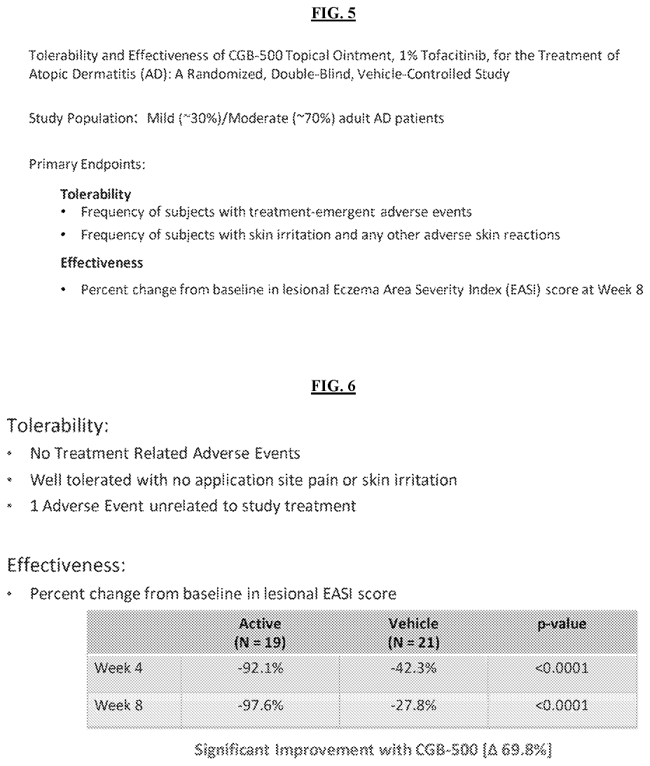

The results were summarized in FIGS. 5-6, showing that the topical tofacitinib compositions disclosed herein are well tolerated and efficacious for treatment of AD.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A topical composition comprising:
from about 1 wt % to about 10 wt % of tofacitinib or a pharmaceutically acceptable salt thereof;
from about 1 wt % to about 30 wt % of an ionic liquid comprising choline cations and geranic acid anions;
from about 10 wt % to about 30 wt % of glycerin;
from about 30 wt % to about 40 wt % of polyethylene glycol; and
from about 1 wt % to about 15 wt % of diethylene glycol monoethyl ether,
wherein topical composition allows a therapeutically effective amount of tofacitinib to reach a target depth within or beyond the skin.

2. The composition of claim 1, wherein a molar ratio of the choline cations to the geranic acid anion is about 1:2.

3. The composition of claim 1, wherein the pharmaceutical composition comprises from about 1 wt % to about 5 wt % of tofacitinib or a pharmaceutically acceptable salt thereof.

4. The composition of claim 1, wherein the pharmaceutical composition comprises from about 1 wt % to about 3 wt % of tofacitinib or a pharmaceutically acceptable salt thereof.

5. The composition of claim 1, wherein the pharmaceutical composition comprises about 1 wt % of tofacitinib or a pharmaceutically acceptable salt thereof.

6. The composition of claim 1, wherein the pharmaceutical composition comprises about 3 wt % of tofacitinib or a pharmaceutically acceptable salt thereof.

7. The topical composition of claim 1, wherein composition comprises tofacitinib.

8. The topical composition of claim 1, wherein the composition comprises tofacitinib citrate.

9. The composition of claim 1, wherein the pharmaceutical composition comprises from about 20 wt % to about 30 wt % of the ionic liquid.

10. The composition of claim 1, wherein the pharmaceutical composition comprises about 25 wt % of the ionic liquid.

11. The composition of claim 1, wherein the composition is a topical ointment.

12. The composition of claim 11, wherein the composition comprises from about 5 wt % to about 20 wt % of propylene glycol.

13. The composition of claim 11, wherein the polyethylene glycol comprises polyethylene glycol 3350.

14. The composition of claim 11, wherein the composition comprises from about 0.01 wt % to about 0.1 wt % of butylated hydroxyanisole.

15. The composition of claim 11, wherein the composition comprises from about 0.1 wt % to about 1 wt % of titanium dioxide.

16. A topical composition comprising:
from about 1 wt % to about 10 wt % of tofacitinib or a pharmaceutically acceptable salt thereof;
from about 1 wt % to about 30 wt % of an ionic liquid comprising choline cations and geranic acid anions;
from about 10 wt % to about 35 wt % of polyethylene glycol; and
from about 10 wt % to about 45 wt % of diethylene glycol monoethyl ether,
wherein topical composition allows a therapeutically effective amount of tofacitinib to reach a target depth within or beyond the skin.

17. The composition of claim 16, wherein the composition is a topical gel.

18. The composition of claim 16, wherein the polyethylene glycol comprises polyethylene glycol 400.

19. The composition of claim 16, wherein the composition comprises from about 5 wt % to about 35 wt % of isopropyl myristate.

20. The composition of claim 16, wherein the composition comprises from about 0.01 wt % to about 0.1 wt % of butylated hydroxyanisole.

21. The composition of claim 16, wherein the composition comprises from about 0.1 wt % to about 20 wt % of Polyamide-3.
